(19) United States
(12) Patent Application Publication
(10) Pub. No.: US 2025/0001410 A1
(43) Pub. Date: Jan. 2, 2025

(54) Systems and Methods for Sample Analysis

(71) Applicant: Abbott Laboratories, Abbott Park, IL (US)

(72) Inventors: Toru Yoshimura, Chiba (JP); Yoshiyuki Arai, Tokyo (JP); Tomotaka Komori, Saitama (JP); Ryotaro Chiba, Chiba (JP); Jeffrey B. Huff, Lincolnshire, IL (US)

(21) Appl. No.: 18/705,941

(22) PCT Filed: Nov. 3, 2022
(86) PCT No.: PCT/US2022/048856

§ 371 Date: Apr. 29, 2024

Related U.S. Application Data
(60) Provisional application No. 63/275,359, filed on Nov. 3, 2021.

(51) Int. Cl. B01L 3/00

(52) U.S. Cl. CPC B01L 3/5025; B01L 2200/0621; B01L 2200/0668; B01L 2200/0689; B01L 2300/0829; B01L 2300/0896; B01L 2300/161; B01L 2400/043

(57) ABSTRACT

Sample analysis systems and methods using assay surfaces, assay processing units (APUs), assay processing systems (APSs), and laboratory systems are disclosed. An assay surface includes a sample processing component comprising a plurality of regions, including at least one wash region and at least one storage region configured to hold a plurality of solid supports moveable through the regions under a magnetic force, and a detection component configured to receive the solid supports. An APU includes an assay surface receiving component, a magnetic element configured to generate a moveable magnetic field, and one or more processors configured to move the magnetic field. An APS includes one or more assay surfaces and an APU. A laboratory system includes one or more APSs and a controller for parallel processing. Sample processing and detection methods are disclosed with a reduced sample volume and/or shortened processing time and/or higher sensitivity.

### CROSS-REFERENCE TO RELATED APPLICATIONS

This application claims the benefit, under 35 U.S.C. § 119 (e), of U.S. Provisional Patent Application No. 63/275,359, filed Nov. 3, 2021, which is incorporated herein by reference in its entirety and for all purposes.

### BACKGROUND

### Field of the Disclosed Subject Matter

The present disclosed subject matter relates to devices, systems, and methods for preparation, detection, and analysis of an analyte of interest in a sample with increased sensitivity and decreased processing time.

### Description of Related Art

Methods and devices that can accurately analyze one or more analytes of interest in a sample can be beneficial for diagnostics, prognostics, environmental assessment, food safety, applications involving detection of chemical or biological agents, and the like. Such methods and devices can be configured for accuracy, precision, and/or sensitivity, as well as to allow individual samples to be analyzed in a shorter amount of time and with reduced instrumentation footprint.

Techniques for sample preparation in a system for sample analysis can include preparing a sample, for example and without limitation by, combining the sample with reagents and/or enzymes in a reaction vessel. In known commercial laboratory systems for sample analysis, sample processing times can take up to 20 minutes or longer to prepare a sample for detection and analysis. The duration of sample preparation time can be due at least in part to lack of suitable automated systems to prepare different samples to perform a variety of different assays. The volume of the sample and/or amount of reagent used to obtain signal suitable for detection can also affect sample preparation time. Additionally, achieving suitable concentrations of analyte within the sensitivity and detection range of conventional detection systems and methods can involve increased incubation or amplification times, further increasing the amount of time to detect an analyte of interest.

Techniques for sample detection in a system for sample analysis can include using or incorporating analog detection systems and methods. The sensitivity and detection range of such analog systems and methods can be a factor in determining the sample size and/or processing times used to achieve a suitable concentration of analyte within the sensitivity and detection range of the sample detection apparatus. As such, there is an interest in methods and devices for sample detection to shorten the processing time and increase the detection sensitivity.

It can also be beneficial for methods and devices for sample detection to be able to prepare a sample in a smaller volume and/or with a shortened sample processing time. Furthermore, it can be beneficial for methods and devices for sample detection to automate the sample processing and detection processes, and to provide high-sensitivity detection of analytes of interest in samples, for example, but not limited to, use in a laboratory environment, such as a clinical or point-of-care laboratory environment.

As such, there remains an opportunity for methods and devices for sample detection that can achieve increased throughput at least in part due to decreased sample preparation time and/or increased sensitivity of the sample processing and detection system.

### SUMMARY

Systems, devices, and methods for analysis of an analyte of interest in a sample are disclosed herein. In accordance with one aspect of the disclosure, an assay surface (AS) for analysis of an analyte interest in a sample and a method using the AS to analyze an analyte of interest are disclosed herein. In accordance with another aspect of the disclosure, an assay processing unit (APU) for performing sample processing and analyte detection on an assay surface and a method using the APU to analyze an analyte of interest are disclosed herein. In accordance with another aspect of the disclosure, an assay processing system (APS) for analysis an analyte of interest in a sample and a method using the APS to analyze an analyte of interest are disclosed herein. In accordance with another aspect of the disclosure, a laboratory system for analysis one or more analytes of interest in a plurality of samples and a method using the laboratory system are disclosed herein. In accordance with another aspect of the disclosure, laboratory systems with a shorter processing time and/or a higher throughput and methods for using such laboratory systems are disclosed.

In accordance with one aspect of the disclosure, an assay surface (AS) can include a sample processing component configured to process the sample for detection, wherein the sample processing component includes a plurality of sample preparation regions, including at least one wash region configured to hold a volume of liquid and at least one storage region configured to hold a plurality of solid supports, wherein the plurality of solid supports is moveable through the plurality of sample preparation regions under a magnetic force; and a detection component configured to receive the plurality of solid supports by the magnetic force and to detect a presence of the analyte or determine a level or concentration of the analyte.

Additionally or alternatively, the plurality of solid supports can be magnetic or paramagnetic microparticles or beads, and can specifically bind to the analyte of interest or at least one reagent or conjugate. Additionally or alternatively, the sample processing component can further include the plurality of solid supports in the at least one storage region. Additionally or alternatively, the sample processing component can further include at least one mixing region configured to mix the plurality of solid supports, the analyte of interest, and at least one reagent or conjugate. Additionally or alternatively, the sample processing component can further include the at least one reagent or conjugate in the at least one mixing region. Furthermore, the at least one mixing region can have a volume capacity of about 25 μL or less.

Additionally or alternatively, at least one reagent can be selected from a group consisting of a detectable label, a binding member, a dye, a surfactant, a diluent, and a combination thereof. Furthermore, the binding member can include a receptor or an antibody.

Additionally or alternatively, the at least one wash region can be configured to wash off any molecules not bound to any solid supports. Furthermore, the at least one wash region has a volume capacity of about 10 μL or less.

Additionally or alternatively, the assay surface can include a plurality of channels, wherein each of the plurality of channels is in between a first and second sample preparation regions. Additionally or alternatively, the assay surface can include a plurality of stopping elements, wherein the assay surface includes a plurality of stopping elements, wherein at least one of the plurality of stopping elements is between the first and second sample preparation regions. Additionally or alternatively, when the at least one stopping element is removed, a volume of liquid in the first region is fluidically connected to a volume of liquid in the second region. Furthermore, after passing the at least one wash region, the plurality of solid supports is moved into the detection component under magnetic force.

Additionally or alternatively, the detection component can be configured for optical detection, analog detection, or digital detection. Furthermore, the detection component can include an array of element, wherein each of the array of element is dimensioned to hold a single one of the plurality of solid supports. Additionally or alternatively, the array of elements can include an array of nanowells. Additionally or alternatively, the detection component can be further configured to receive a substrate fluid, the substrate fluid configured to provide a detectable signal from the plurality of solid supports, and the detection component can be further configured to receive an inert liquid, the inert liquid configured to seal each element of the array of elements containing a portion of the substrate fluid and a single one of the plurality of solid supports. Furthermore, after the plurality of solid supports is moved into the detection component, the detection component can be configured to obtain images of the array of elements. Additionally or alternatively, the detection component can be configured for single-molecule counting.

Additionally or alternatively, the assay surface includes a hydrophobic material. Additionally or alternatively, the assay surface can further include a plurality of volumes of liquids, a plurality of solid supports, and at least one reagent or conjugate in the plurality of sample preparation regions.

In accordance with the aspect of the disclosure, a method for analysis of an analyte of interest in a sample using the assay surface can include loading at least one volume of liquid into at least one wash region of the assay surface, wherein the assay surface includes: a sample processing component configured to process the sample for detection, wherein the sample processing component includes a plurality of sample preparation regions, including the at least one wash region configured to hold a volume of liquid and at least one storage region configured to hold a plurality of solid supports, wherein the plurality of solid supports is moveable through the plurality of sample preparation regions under a magnetic force; and a detection component configured to receive the plurality of solid supports by the magnetic force and to detect a presence of the analyte or determine a level or concentration of the analyte; loading at least one volume of liquid into the detection component; loading a volume of liquid comprising the analyte into the sample processing component; and detecting the analyte of interest in the detection component. The assay surface used can include any assay surface disclosed herein.

Additionally or alternatively, when the sample processing component includes a plurality of solid supports, the method can further include moving the plurality of solid supports through the plurality sample preparation regions into the detection component under the magnetic force before detecting the analyte of interest in the detection component.

Additionally or alternatively, the method further includes: loading a plurality of solid supports onto the sample processing component, and moving the plurality of solid supports through the plurality sample preparation regions into the detection component under the magnetic force before detecting the analyte of interest in the detection component.

In accordance with another aspect of the disclosure, an assay processing unit (APU) for performing sample processing and analyte detection on an assay surface comprising a sample processing component and a detection component are disclosed herein. The APU can include: an assay surface receiving component configured to receive and hold an assay surface; a magnetic element configured to generate a magnetic field, wherein the magnetic field is movable along the assay surface when received by the receiving component; and one or more processors configured to move the magnetic field to urge at least one solid support disposed on the assay surface through at least one volume of liquid in at least one region of the sample processing component and to the detection component of the assay surface using the magnetic field.

Additionally or alternatively, the magnetic element can be a magnet. Additionally or alternatively, the APU can include a sliding element, for example, a motor, configured to move the magnetic element under the control of the one or more processors along a horizontal direction of a plane defined by a top surface of the assay surface when received by the receiving component. Additionally or alternatively, the APU can include a drive element, for example, a motor or a string, configured to move the magnetic element under the control of the processor in a perpendicular direction to a plane defined by a top surface of the assay surface when received by the receiving component. Additionally or alternatively, the magnetic element can include an electromagnet configured to generate a movable magnetic field. Additionally or alternatively, the APU can include a mixing dynamics element, for example, a vibration motor or an electromagnet, controlled by the one or more processors configured to cause at least one volume of liquid in at least one region of the assay surface when received by the receiving component to mix under a predetermined frequency. Additionally or alternatively, the one or more processors can cause the detection component of the assay surface when received by the receiving component to obtain images of the detection component.

In accordance with the aspect of the disclosure, a method for performing sample processing and analyte detection on the assay surface comprising a sample processing component and a detection component using the APU, can include: receiving an assay surface into an assay surface receiving component of the APU; generating a magnetic field by a magnetic element of the APU, wherein the magnetic field is movable along the assay surface; and detecting the analyte of interest in the detection component controlled by the one or more processors of the APU.

Additionally or alternatively, when the assay surface includes a plurality of solid supports, the method can further include moving the magnetic field controlled by one or more processors of the APU to urge at least one solid support disposed on the assay surface through at least one volume of liquid in at least one region of the sample processing component and to the detection component of the assay surface using the magnetic field before detecting the analyte of interest in the detection component.

Additionally or alternatively, the method further includes: loading a plurality of solid supports onto the sample processing component, and moving the magnetic field controlled by one or more processors of the APU to urge at least one solid support disposed on the assay surface through at least one volume of liquid in at least one region of the sample processing component and to the detection component of the assay surface using the magnetic field before detecting the analyte of interest in the detection component. The method can be used with any assay surfaces or APUs disclosed herein.

In accordance with another aspect of the disclosure, an assay processing system (APS) for analysis an analyte of interest in a sample is disclosed. The APS can include: one or more assay surfaces, wherein at least one assay surface includes: a sample processing component configured to process the sample for detection, wherein the sample processing component includes a plurality of sample preparation regions, including at least one wash region configured to hold a volume of liquid and at least one storage region configured to hold a plurality of solid supports, wherein the plurality of solid supports is moveable through the plurality of sample preparation regions under a magnetic force; and a detection component configured to receive the plurality of solid supports by the magnetic force and to detect a presence of the analyte or determine a level or concentration of the analyte; and an assay processing unit (APU) comprising: an assay surface receiving component configured to receive and hold the one or more assay surface; a magnetic element configured to generate a magnetic field, wherein the magnetic field is movable along at least one assay surface when received by the receiving component; one or more processors configured to move the magnetic field to urge at least one solid support disposed on the at least one assay surface through at least one volume of liquid in at least one region of the sample processing component and to the detection component of the assay surface using the magnetic field.

Additionally or alternatively, the APS can include any suitable assay surface in accordance with the disclosed subject matter. Additionally or alternatively, the APS can include any suitable APU in accordance with the disclosed subject matter.

In accordance with the aspect of the disclosure, a method for analysis of an analyte of interest in a sample using an assay processing system (APS) comprising an assay surface and an assay processing unit (APU), including: loading at least one volume of liquid into at least one wash region of the assay surface, wherein the assay surface comprising: a sample processing component configured to process the sample for detection, wherein the sample processing component includes a plurality of sample preparation regions, including the at least one wash region configured to hold a volume of liquid and at least one storage region configured to hold a plurality of solid supports, wherein the plurality of solid supports is moveable through the plurality of sample preparation regions under a magnetic force; and a detection component configured to receive the plurality of solid supports by the magnetic force and to detect a presence of the analyte or determine a level or concentration of the analyte; loading at least one volume of liquid into the detection component; loading a volume of liquid comprising the analyte into the sample processing component; receiving the assay surface into an assay surface receiving component of the APU; generating a magnetic field by a magnetic element of the APU, wherein the magnetic field is movable along the assay surface; and detecting the analyte of interest in the detection component controlled by the one or more processors of the APU. Additionally or alternatively, the one or more assay surfaces used in the method can include an assay surface in accordance with the disclosed subject matter. Additionally or alternatively, the APU used in the disclosed method can include an APU in accordance with the disclosed subject matter.

Additionally or alternatively, when the at least one assay surface includes a plurality of solid supports, the method further includes moving the magnetic field controlled by the one or more processors of the APU to urge at least one solid support disposed on the assay surface through at least one volume of liquid in at least one region of the sample processing component and to the detection component of the assay surface using the magnetic field before detecting the analyte.

Additionally or alternatively, the method can further include: loading a plurality of solid supports onto the assay surface, and moving the magnetic field controlled by the one or more processors of the APU to urge at least one solid support disposed on the assay surface through at least one volume of liquid in at least one region of the sample processing component and to the detection component of the assay surface using the magnetic field before detecting the analyte.

In accordance with another aspect of the disclosure, a laboratory system for analysis of one or more analytes of interest in a plurality of samples is disclosed. The laboratory system can include: one or more assay processing systems (APSs), wherein at least one APS includes: one or more assay surfaces, wherein at least one assay surface includes: a sample processing component configured to process the sample for detection, wherein the sample processing component includes a plurality of sample preparation regions, including at least one wash region configured to hold a volume of liquid and at least one storage region configured to hold a plurality of solid supports, wherein the plurality of solid supports is moveable through the plurality of sample preparation regions under a magnetic force; and a detection component configured to receive the plurality of solid supports by the magnetic force and to detect a presence of the analyte or determine a level or concentration of the analyte; and an assay processing unit (APU) comprising: an assay surface receiving component configured to receive and hold the one or more assay surface; a magnetic element configured to generate a magnetic field, wherein the magnetic field is movable along at least one assay surface when received by the receiving component; one or more processors configured to move the magnetic field to urge at least one solid support disposed on the at least one assay surface through at least one volume of liquid in at least one region of the sample processing component and to the detection component of the assay surface using the magnetic field; and a controller configured to control a plurality of the one or more APSs to process a corresponding sample and to detect a presence of at least one corresponding analyte or determine a level or concentration of the at least one corresponding analyte substantially in parallel.

Additionally or alternatively, the one or more APSs can include an APS in accordance with the disclosed subject matter. The one or more assay surfaces can include any assay surface as disclosed herein. Additionally or alternatively, the APU can include any APU as disclosed herein.

Additionally or alternatively, the laboratory system is configured to perform one or more of an HIV p24 assay, an HBsAg assay, a Troponin I assay, a TSH assay, a Myoglobobin assay, a PSA assay, a BNP assay, a PIVKA-II assay, an HIV Ab assay, an estradiol assay, and a COVID-Ag assay. Additionally or alternatively, the laboratory system has a throughput of at least 360 samples per hour. Additionally or alternatively, the laboratory system has a throughput of at least 375 of the samples per hour per square meter footprint of the laboratory system.

In accordance with the aspect of the disclosure, a method for using the laboratory system can include: loading at least one volume of liquid into at least one wash region of the assay surface, wherein the assay surface comprising: a sample processing component configured to process the sample for detection, wherein the sample processing component includes a plurality of sample preparation regions, including the at least one wash region configured to hold a volume of liquid and at least one storage region configured to hold a plurality of solid supports, wherein the plurality of solid supports is moveable through the plurality of sample preparation regions under a magnetic force; and a detection component configured to receive the plurality of solid supports by the magnetic force and to detect a presence of the analyte or determine a level or concentration of the analyte; loading at least one volume of liquid into the detection component; loading a volume of liquid comprising the analyte into the sample processing component; receiving the assay surface into an assay surface receiving component of the APU; generating a magnetic field by a magnetic element of the APU, wherein the magnetic field is movable along the at least one assay surface; and detecting the analyte of interest in the detection component controlled by one or more processors of the corresponding APU, wherein the controller is configured to control a plurality of the one or more APSs to perform corresponding steps for a corresponding sample and to detect a presence of at least one corresponding analyte or determine a level or concentration of the at least one corresponding analyte substantially in parallel.

Additionally or alternatively, when the at least one assay surface includes a plurality of solid supports, the method can further include moving the magnetic field controlled by the one or more processors of the APU to urge at least one solid support disposed on the assay surface through at least one volume of liquid in at least one region of the sample processing component and to the detection component of the assay surface using the magnetic field before detecting the analyte.

Additionally or alternatively, the method can further include: loading a plurality of solid supports onto the at least one assay surface, and moving the magnetic field controlled by the one or more processors of the APU to urge at least one solid support disposed on the assay surface through at least one volume of liquid in at least one region of the sample processing component and to the detection component of the assay surface using the magnetic field before detecting the analyte.

Additionally or alternatively, the method can use an assay surface or an APU according to the disclosed subject matter. Additionally or alternatively, the method can perform on one or more of an HIV p24 assay, an HBsAg assay, a Troponin I assay, a TSH assay, a Myoglobobin assay, a PSA assay, a BNP assay, a PIVKA-II assay, an HIV Ab assay, an estradiol assay, and a COVID-Ag assay. Additionally or alternatively, the method can be used on the laboratory system which has a throughput of at least 360 samples per hour. Additionally or alternatively, the method can be used on the laboratory system which has a throughput of at least 375 of the samples per hour per square meter footprint of the laboratory system.

In accordance with another aspect of the disclosure, a laboratory system for high-throughput analysis of an analyte of interest in a sample can include a sample processing component configured to process a sample for detection, wherein the sample processing component is configured to obtain a level or a concentration of an analyte in the sample, or a level or a concentration of a conjugate indicative of the analyte in the sample, suitable for detection, and a detection component configured to detect a presence of the analyte in the sample. The laboratory system can have a time-to-result of less than 6 minutes, or a time-to-result within a range of 3 to 5 minutes or a time-to-result within a range of 3 to 7 minutes. Additionally or alternatively, the laboratory system can have a throughput of at least about 360 samples per hour. In addition, or as a further alternative, the laboratory system can have a throughput of at least about 375 samples per hour per square meter of the laboratory system, or within a range of 375 to 600 samples per hour per square meter footprint of the laboratory system.

Methods for high-throughput analysis of an analyte of interest in a sample are also provided. Such methods include processing a sample for detection, including obtaining a level or a concentration of an analyte in the sample, or a level or a concentration of a conjugate indicative of the analyte in the sample, suitable for detection, and detecting a presence of the analyte in the sample. Processing the sample and detecting the presence of the analyte in the sample are completed for the sample in less than 6 minutes, or within a range of 3 to 5 minutes, or within a range of 3 to 7 minutes. Additionally or alternatively, processing the sample and detecting the presence of the analyte in the sample are completed for at least about 360 samples per hour. In addition, or as a further alternative, processing the sample and detecting the presence of the analyte in the sample are completed for at least about 375 of the samples per hour per square meter of the laboratory system, or within a range of 375 to 600 samples per hour per square meter footprint of the laboratory system

### BRIEF DESCRIPTION OF THE DRAWINGS

FIG. 1 is a diagram illustrating an exemplary assay surface for sample analysis, including a sample processing component and a detection component in accordance with the disclosed subject matter.

FIG. 2 is a diagram illustrating an exemplary embodiment of a detection component in accordance with the disclosed subject matter.

FIG. 3 is a chart illustrating exemplary noise level performance of an analog detection system and a digital detection system for purpose of comparison with and confirmation of the disclosed subject matter.

FIG. 4 is a chart illustrating exemplary sensitivity characteristics of an exemplary assay surface with a digital detection component in accordance with the disclosed subject matter compared to systems using analog detection.

FIGS. 5A-5D are charts illustrating exemplary sensitivity characteristics of an exemplary assay surface with a digital detection component in accordance with the disclosed subject matter compared to systems using analog detection.

FIG. 6 is a chart illustrating additional data about exemplary sensitivity performance using an exemplary assay surface with a digital detection component for sample analysis in accordance with the disclosed subject matter to perform an HIV p24 assay compared to systems using analog detection.

FIG. 7A-7C are charts illustrating exemplary sensitivity and dynamic range characteristics of an exemplary assay surface with a digital detection component for sample analysis in accordance with the disclosed subject matter to perform an estradiol assay compared to systems using analog detection.

FIG. 8 is a chart illustrating exemplary sensitivity and processing time characteristics of an exemplary assay surface with a digital detection component in accordance with the disclosed subject matter compared to systems using analog detection.

FIG. 9 is a chart illustrating intensity characteristics during an enzyme reaction using an exemplary assay surface for sample analysis in accordance with the disclosed subject matter.

FIG. 10 is a diagram illustrating exemplary detection techniques for an assay surface for sample analysis in accordance with the disclosed subject matter.

FIGS. 11A and 11B are charts illustrating exemplary dynamic range characteristics of an exemplary assay surface for sample analysis with digital detection in accordance with the disclosed subject matter compared to systems using analog detection.

FIG. 12 is a diagram illustrating an exemplary assay surface in plan view for use with an assay processing unit (APU) for sample analysis in accordance with the disclosed subject matter.

FIG. 13 is a diagram illustrating movements of microparticles or beads through volumes of liquid using a moving magnetic field in an exemplary assay surface in accordance with the disclosed subject matter.

FIG. 14 is an image showing alternative embodiments of assay surfaces for use with an assay processing unit (APU), an assay processing system (APS), or a laboratory system for sample analysis in accordance with the disclosed subject matter.

FIG. 15 is a diagram illustrating an alternative embodiment of an assay surface for use with an APU, an APS, or a laboratory system for sample analysis in accordance with the disclosed subject matter.

FIGS. 16A and 16B are charts illustrating details of an exemplary wash process in an assay surface for purpose of comparison with systems using conventional sample preparation components.

FIG. 17 is a chart illustrating characteristics of an exemplary assay surface for sample analysis in accordance with the disclosed subject matter compared to conventional systems for sample analysis.

FIG. 18 is a chart illustrating details of an exemplary laboratory system using one or more assay surfaces for sample analysis in accordance with the disclosed subject matter compared to conventional systems for sample analysis.

FIG. 19 is a diagram illustrating additional details of an exemplary APU for an exemplary laboratory system for sample analysis in accordance with the disclosed subject matter.

FIG. 20 is a diagram illustrating an alternative embodiment of an assay surface for sample analysis in accordance with the disclosed subject matter.

FIG. 21 is a diagram illustrating an exploded view of an exemplary assay processing system (APS) with an APU and an exemplary assay surface for sample preparation and detection.

FIG. 22 is a diagram illustrating a side view of the exemplary APS for sample preparation and detection of FIG. 21.

FIGS. 23A and 23B are diagrams illustrating exemplary wash techniques in a wash region of an exemplary assay surface using the exemplary APS of FIG. 21.

FIGS. 24A-24D are diagrams illustrating an assembly of an alternative embodiment of an assay surface including a plurality of stopping elements.

FIG. 25 is a diagram illustrating an alternative embodiment of an assay surface including a plurality of stopping elements.

FIG. 26 is a chart illustrating a wash efficiency of HIV Ag p24 assay using the exemplary wash technique in the exemplary APS of FIG. 21 in accordance with the disclosed subject matter compared to a King-Fisher wash technique.

FIGS. 27A-27B are diagrams illustrating exemplary bead loading techniques to load beads or microparticles into an exemplary detection region of an assay surface according to the disclosed subject matter.

### DETAILED DESCRIPTION

Reference will now be made in detail to the various exemplary embodiments of the disclosed subject matter, exemplary embodiments of which are illustrated in the accompanying drawings. The structure and corresponding method of operation of the disclosed subject matter will be described in conjunction with the detailed description of the system.

The systems and methods presented herein can be used for detection of an analyte of interest in a sample, including but not limited to samples for analysis in a laboratory environment. For purpose of illustration and not limitation, the sample can include a biological fluid sample, for example and as embodied herein, a sample of blood, plasma, serum, saliva, sweat, urine, or any other sample suitable for analysis using the systems and techniques described herein. As embodied herein, the systems and techniques for sample analysis described herein can analyze a single sample in about 5 minutes or less. Additionally or alternatively, as embodied herein, the systems and techniques for sample analysis described herein can have a throughput to analyze at least about 360 samples per hour, and more preferably at least about 375 samples per hour per square meter, or within a range of about 375 to 600 samples per hour per square meter.

According to aspects of the disclosed subject matter, exemplary sample analysis systems are provided in conjunction with exemplary methods for sample analysis. Exemplary sample analysis systems and methods can use exemplary assay surfaces, assay processing units (APUs), assay processing systems (APSs), and laboratory systems for sample processing and detection. For example and as embodied herein, exemplary sample analysis systems and methods can be used to perform any type of assay, including, but not limited to, an immunoassay, such as sandwich immunoassay (e.g., monoclonal-polyclonal sandwich immunoassays), including enzyme detection (e.g., enzyme immunoassay (EIA) or enzyme-linked immunosorbent assay (ELISA)), competitive inhibition immunoassay (e.g., forward and reverse), enzyme multiplied immunoassay technique (EMIT), a competitive binding assay, bioluminescence resonance energy transfer (BRET), one-step antibody detection assay, homogeneous assay, heterogeneous assay, capture on the fly assay, or any other immunoassay.

For purpose of illustration and not limitation, as embodied herein, a detectable label, such as one or more fluorescent labels or tags, can be attached to an analyte for detection. Additionally or alternatively, other detectable labels, such as one or more labels or tags attached by a cleavable linker, which can be cleaved, for example, chemically or by photocleavage, can be attached to a detection antibody.

For purpose of illustration and not limitation, “bead,” “particle,” and “microparticle” are used herein interchangeably and refer to a substantially spherical solid support. “Magnetic bead” and “paramagnetic bead” refer to a substantially spherical solid support that can be facilitated under magnetic force. For purpose of illustration and not limitation, “chip,” “reaction chip,” and “sample chip” are used herein interchangeably and refer to an assay surface for analysis of an analyte of interest in a sample in accordance with the disclosed subject matter.

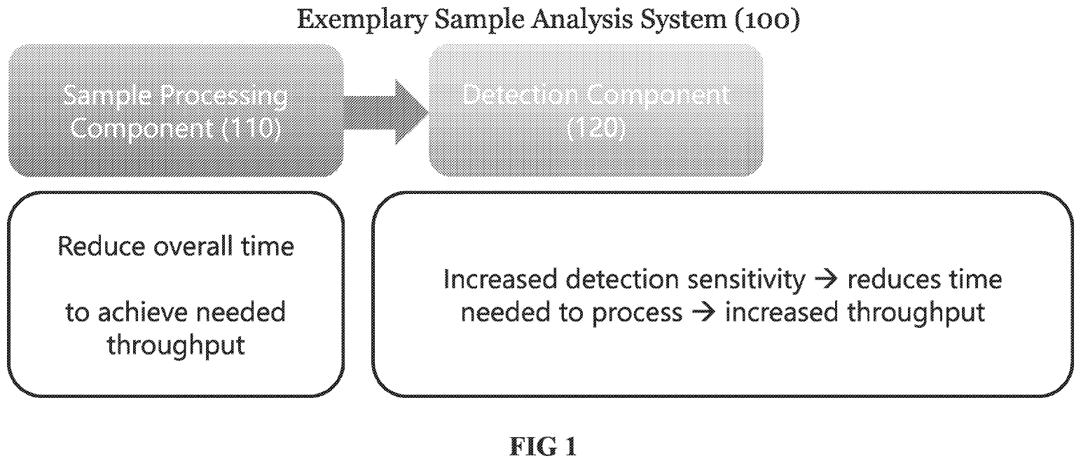

FIG. 1 illustrates an exemplary sample assay surface (100) according to the disclosed subject matter. As disclosed herein, an exemplary system for sample analysis generally includes two components: a sample processing component (110) and a detection component (120). Sample processing component (110) can be configured to prepare a sample for analysis and/or detection, which can include, for example and without limitation, purifying a sample of interest, isolating an analyte of interest in the sample, and/or combining the sample with reactive elements, such as conjugates, enzymes, reagents, diluents, microparticles, or other elements used to perform the analysis and/or detection of interest. For purpose of illustration and not limitation, sample processing component (110) can be configured to process an analyte of interest in the sample, or a detectable component of the sample, such as a conjugate, to have a level or concentration suitable for detection by the assay surface (100). Detection component (120) is configured to detect or analyze the analyte of interest in the sample. While exemplary sample analysis systems are described herein using optical-based detection components, any suitable detection component can be used, for example and without limitation, electrical detection, electrochemical detection, viscoelastic detection, or any other suitable detection techniques. If optical detection is used, such optical detection can use, for purpose of illustration and not limitation, digital detection techniques, analog detection techniques, or a combination of digital and analog detection techniques.

As embodied herein, sample processing component (110) can be configured to prepare the sample using any suitable sample preparation techniques. For purpose of illustration and not limitation, sample preparation components can be configured to isolate and/or purify an analyte of interest in the sample. For example and without limitation, sample preparation components can include manual pipetting, including, but not limited to, using one or more pipettes to move a sample into a reaction location, combine one or more reactive elements with the sample, and/or wash the sample. Additionally or alternatively, automatic pipetting systems can be used to perform any or all sample preparation by the sample preparation component. In addition, or as a further alternative, and as embodied herein, sample preparation components can be configured to perform sample preparation process steps wherein, and for purpose of illustration and not limitation, particles or beads are passed through the surface of a liquid and/or through an air-aqueous or oil-aqueous boundary.

For purpose of illustration and not limitation, as embodied herein, a heterogeneous format can be used. For example, after the test sample is obtained from a subject, a first mixture can be prepared. As embodied herein, the mixture can include the test sample being assessed for analyte of interest and a first specific binding partner. The first specific binding partner and any analyte of interest in the test sample can be combined to form a first specific binding partner-analyte of interest complex. As embodied herein, the first specific binding partner can be an anti-analyte of interest antibody or a fragment thereof. The order in which the test sample and the first specific binding partner are added to form the mixture can be reversed. As embodied herein, the first specific binding partner can be immobilized on a solid phase. The solid phase used in the immunoassay (e.g., for the first specific binding partner and, optionally, the second specific binding partner) can be any solid phase, such as, but not limited to, a magnetic particle, a bead, a nanobead, a microbead, a nanoparticle, a microparticle, a membrane, a scaffolding molecule, a film, a filter paper, a disc, or a chip (e.g., a microfluidic chip). For purpose of illustration and not limitation, as embodied herein, sample processing can include incubating the sample and the first binding member, for example, after mixing, for a period suitable to allow for the binding interaction between the binding member and analyte to occur. As embodied herein, the incubating can be in a binding buffer that facilitates the specific binding interaction. The binding affinity and/or specificity of the first binding member and/or the second binding member can be manipulated or altered in the assay, for example and without limitation, by varying the binding buffer. For example, and as embodied herein, the binding affinity and/or specificity can be increased or decreased by varying the binding buffer.

After the mixture including the first specific binding partner-analyte of interest complex is formed, including before or after any incubation (if performed) any unbound analyte of interest can be removed from the complex using any suitable technique. For example and without limitation, the unbound analyte of interest can be removed by washing. For purpose of illustration not limitation, as embodied herein, the disclosed systems and methods can perform one-step or two-step assay preparations. As embodied herein, the first specific binding partner can be present in excess of any analyte of interest present in the test sample, such that all analyte of interest that is present in the test sample can be bound by the first specific binding partner.

After any unbound analyte of interest is removed, for purpose of illustration and as embodied herein, a second specific binding partner can be added to the mixture to form a first specific binding partner-analyte of interest-second specific binding partner complex. The second specific binding partner can be an anti-analyte of interest (such as an antibody) that binds to an epitope on analyte of interest that differs from the epitope on analyte of interest bound by the first specific binding partner. Additionally or alternatively, the second specific binding partner can be labeled with or contain a detectable label (e.g., a fluorescent label, a tag attached by a cleavable linker, or any other suitable label).

Additionally or alternatively, and as embodied herein, immobilized antibodies or fragments thereof can be incorporated into the immunoassay. The antibodies can be immobilized onto any suitable support, such as, but not limited to, magnetic or chromatographic matrix particles, latex particles or modified surface latex particles, polymer or polymer film, plastic or plastic film, planar substrate, a microfluidic surface, or pieces of a solid substrate material.

Sample processing can include additional or alternative steps to obtain a level or concentration of analyte or conjugate suitable for detection, for example, an amplification component. For example, amplification or lysis can be performed, such as, but without limitation, if the assay involves a molecular process. For purpose of illustration and not limitation, amplification can be performed using any suitable amplification technique, including isothermal amplification and polymerase chain reaction (PCR) amplification. For example only and not limitation, amplification can be performed using transcription mediated amplification (TMA), recombinase polymerase amplification (RPA), or any suitable isothermal amplification technique.

In additional, or as a further alternative, and as embodied herein, detection component (120) can be configured to detect or analyze an analyte of interest in the sample, including, but not limited to, detecting the presence or absence of the analyte and/or determining a concentration of the analyte in the sample. For purpose of illustration and not limitation, detection components can perform detection using optical detection, which can include analog detection, digital detection, illumination detection, fluorescence detection, or any combination of these techniques. Additionally or alternatively, the detection component can be configured to perform single-molecule counting.

Sensitivity of the detection component can affect other characteristics of the sample analysis system affecting overall performance of the system, as discussed further herein. As used herein, “sensitivity” of the detection component refers to a level or a concentration of an analyte of interest in a sample (or a conjugate, if used) that can be detected by the detection component (120), where a lower level or concentration that can be detected indicates a higher sensitivity. For example and without limitation, increasing the sensitivity of the detection component (120) can allow for detection of a lower concentration of analytes in a sample, which can reduce the time involved to process the analyte of interest to obtain a concentration of the analyte (or conjugate if used) suitable for detection compared to conventional systems.

Additionally or alternatively, increasing the sensitivity of the detection component can allow for detection to be performed using less sample volume, less reagent or conjugate material, fewer particles or beads, or any combination of these, to obtain an analyte concentration suitable for detection in a similar or faster time compared to conventional systems. For purpose of illustration not limitation, reagents can be selected from a group consisting of a detectable label, a binding member, a dye, a surfactant, a diluent, and a combination thereof. Binding members, if used, can be a receptor or an antibody. In this manner, sample preparation time can be improved due at least in part to less sample manipulation involved and/or improved kinetics of reactions achieved using a lower sample volume, less reagent or conjugate material, and/or fewer particles or beads to obtain an analyte concentration suitable for detection. As such, the time to perform an assay, the cost of materials used for an assay, and/or the amount of sample material (e.g., bodily fluid or organic matter) to be collected to perform an assay can be reduced using a detection component with increased sensitivity.

For purpose of illustration only but not limitation, additional details of systems and methods for sample analysis according to the disclosed subject matter, including exemplary sample processing and detection components, are described in U.S. Patent Application Publication Nos. 2018/0095067, 2018/0104694, and 2018/0188230, each of which is incorporated by reference herein in its entirety.

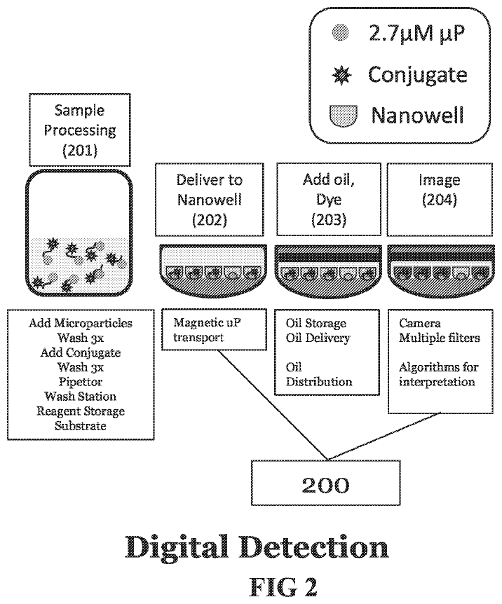

FIG. 2 illustrates exemplary detection components 120 according to the disclosed subject matter. With reference to FIG. 2, for purpose of illustration but not limitation, an exemplary digital detection component (200) is shown. As embodied herein, prior to entering digital detection component (200), at (201), sample processing is performed to obtain a concentration of the analyte (or conjugate if used) suitable for detection. Sample processing can include any combination of steps described herein. For example, a support medium, including, but not limited to, microparticles, beads, or other labels, can be mixed with the sample. As embodied herein, reagents including antibodies and coated microparticles can be combined. The solution can be washed, for example to remove excess reagents and/or unbound microparticles. Any suitable number of washes can be performed for each washing step, including one, two, or three or more washes, and each wash can be performed in a single chamber or location or among different chambers or locations. For example and without limitation, as embodied herein, three washes can be performed. A conjugate can be added to bind with an analyte of interest in the sample. For example and without limitation, the conjugate can include one or more reagents or enzymes selected or configured to react with the analyte of interest to produce a signal for detection by the detection component. The solution with conjugate added can be washed, for example to remove excess conjugate unbound to the analyte of interest. Any suitable number of washes can be performed for each washing step, including one, two, or three or more washes, and each wash can be performed in a single chamber or location or among different chambers or locations. For example and without limitation, as embodied herein, three washes can be performed. Microparticles bound with analytes and conjugates can be added to a substrate for detection. For purpose of illustration and not limitation, the substrate can include a detection region. The microparticles can be added to the substrate using any suitable technique, including but not limited to pipetting, magnetic force or dielectrophoresis. As embodied herein, the detection region can include one or more nanowells.

At (200), digital detection is performed. For example, and as embodied herein, at (202), the microparticles can be moved to the detection region, for example and as embodied herein, an array of nanowells. The microparticles can be moved to the nanowells using any suitable technique, including, but not limited to, pipetting, magnetic force or dielectrophoresis. At (203), an hydrophobic liquid, for example, an oil can be added to seal the nanowells to prevent, among other things, migration of beads or evaporation of the aqueous fluid in the nanowells. For purpose of illustration only, the added oil can be mineral oil, or any other kind of suitable oil. Additionally or alternatively, other suitable hydrophobic liquids can be added to seal the nanowells. Additionally or alternatively, a dye or contrast agent can be added to increase contrast or otherwise improve optical conditions for detection of the analyte of interest in the nanowells. Methods of using a dye in signal-generating digital assays are disclosed, for example and without limitation, in International Patent Application Publication No. WO 2018/143478, which is incorporated by reference herein in its entirety. At (204), one or more images of the microparticles is taken and analyzed to determine the presence or absence of the analyte of interest and/or a concentration of the analyte of interest in the sample.

Digital detection components and methods can significantly increase detection sensitivity in systems for sample analysis compared to systems using analog detection. As such, detection can be performed using a lower concentration of analyte, which can allow for decreased time to process the sample for detection. Additionally or alternatively, detection can be performed using a smaller sample volume, less reagent material, less conjugate material, fewer microparticles, or any combination of these, which can reduce costs to perform each assay. As such, and as described herein, sample preparation time can be improved due at least in part to less sample manipulation involved (e.g., faster washing times) and/or improved kinetics of reactions achieved using a lower sample volume, less reagent or conjugate material, and/or fewer particles or beads to obtain an analyte concentration suitable for detection. Assays using less sample volume and/or reagent material can be performed using smaller equipment, which can reduce the footprint of the laboratory system for performing the assays as discussed further herein. In addition, or as a further alternative, increased detection sensitivity can provide additional benefits when used with multiplexing. For example, and without limitation, when multiple analytes and corresponding signals are combined into a single, multiplexed assay, a noise level associated with the detection of each analyte signal can be multiplied to obtain a total noise level of the multiplexed system. By increasing the detection sensitivity of each signal being detected, the improved sensitivity can be multiplied to further reduce the total noise level of the multiplexed system.

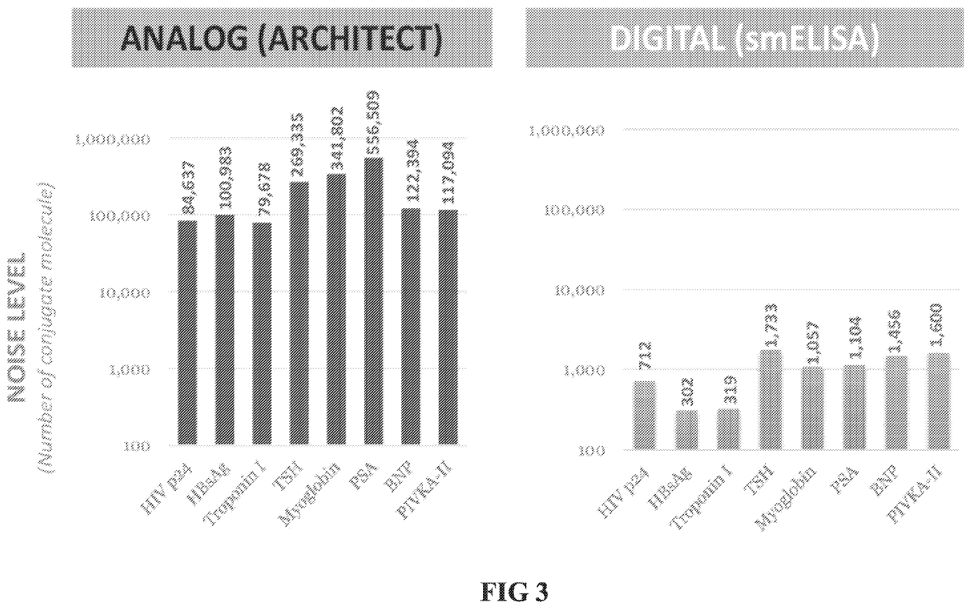

Digital detection can provide increased sensitivity due at least in part to a reduction of noise during detection relative to the signal being measured, for example, producing a higher signal-to-noise ratio. FIG. 3 is a chart illustrating detection noise levels of an exemplary digital detection system of the disclosed subject matter compared with a sample analysis system using analog detection (e.g., Abbott ARCHITECT™ family of systems) for purpose of illustration and confirmation of the disclosed subject matter. For example only, and not limitation, the assays illustrated in FIG. 3 were performed as follows. For ARCHITECT™ HIV Ag/Ab Combo assay (p24 assay) on ARCHITECT™, 100 μL of negative sample (as “0” concentration sample) was applied for a first 18-minute immunoreaction and a second 4-minute immunoreaction. For purpose of illustration not limitation, wash processes can take additional time. For example and as embodied herein, the first immunoreaction can be used for analysis of molecules using microparticles, and the second immunoreaction can be used to detect antigens with second antibodies. The number of conjugate molecules was calculated from relative light unit (RLU) values of chemiluminescence. For Digital HIV p24 assay, 100 μL of negative sample (as “0” concentration sample) was applied for a total of an 18-minute immunoreaction time assay. The number of conjugate molecules was calculated by counting the digital signals.

For ARCHITECT™ HBsAg assay, 75 μL of negative sample (as “0” concentration sample) was applied for a total of 22-minute (18 minutes of immunoreaction and 4 minutes of enzyme reaction) immunoreaction time assay. The number of conjugate molecules was calculated from relative light unit (RLU) values of chemiluminescence. For Digital HBsAg assay, 75 μL of negative sample (as “0” concentration sample) was applied for a total of 18-minute immunoreaction time assay. The number of conjugate molecules was calculated by counting the digital signals.

For ARCHITECT™ Troponin I assay, 150 μL of negative sample (as “0” concentration sample) was applied for a total of 8-minute (4 minutes of immunoreaction and 4 minutes of enzyme reaction) immunoreaction time assay. For purpose of illustration not limitation, wash processes can take additional time. The number of conjugate molecules was calculated from relative light unit (RLU) values of chemiluminescence. For Digital Troponin I assay, 100 μL of negative sample (as “0” concentration sample) was applied for a total of 8-minute immunoreaction time assay. The number of conjugate molecules was calculated by counting the digital signals.

For ARCHITECT™ TSH assay, 150 μL of negative sample (as “0” concentration sample) was applied for a total of 22-minute (18 minutes of immunoreaction and 4 minutes of enzyme reaction) immunoreaction time assay. For purpose of illustration not limitation, wash processes can take additional time. The number of conjugate molecules was calculated from relative light unit (RLU) values of chemiluminescence. For Digital TSH assay, 110 μL of negative sample (as “0” concentration sample) was applied for a total of 18-minute immunoreaction time assay. The number of conjugate molecules was calculated by counting the digital signals.

For ARCHITECT™ Myoglobin assay, 20 μL of negative sample (as “0” concentration sample) was applied for a total of 8-minute (4 minutes of immunoreaction and 4 minutes of enzyme reaction) immunoreaction time assay. For purpose of illustration not limitation, wash processes can take additional time. The number of conjugate molecules was calculated from relative light unit (RLU) values of chemiluminescence. For Digital Myoglobin assay, 20 μL of negative sample (as “0” concentration sample) was applied for a total of 8-minute immunoreaction time assay. The number of conjugate molecules was calculated by counting the digital signals.

For ARCHITECT™ PSA assay, 50 μL of negative sample (as “0” concentration sample) was applied for a total of 22-minute (18 minutes of immunoreaction and 4 minutes of enzyme reaction) immunoreaction time assay. For purpose of illustration not limitation, wash processes can take additional time. The number of conjugate molecules was calculated from relative light unit (RLU) values of chemiluminescence. For Digital PSA assay, 50 μL of negative sample (as “0” concentration sample) was applied for a total of 18-minute immunoreaction time assay. The number of conjugate molecules was calculated by counting the digital signals.

For ARCHITECT™ PIVKA-II assay, 30 μL of negative sample (“0” concentration sample) was applied for a total of 22-minute (18 minutes of immunoreaction and 4 minutes of enzyme reaction) immunoreaction time assay. For purpose of illustration not limitation, wash processes can take additional time. The number of conjugate molecules was calculated from relative light unit (RLU) values of chemiluminescence. For Digital PIVKA-II assay, 30 μL of negative sample (as “0” concentration sample) was applied for a total of 26-minute immunoreaction time assay. For example, and as embodied herein, during the 26-minute immunoreaction time, 18 minutes can involve a first reaction and 8 minutes can involve a second reaction to reduce variations in the assay process. The number of conjugate molecules was calculated by counting the digital signals.

Referring still to FIG. 3, the noise level of the detection correlates to the number of conjugate molecules. On the left side of the chart, for assays performed by the sample analysis system using analog detection, including HIV p24, HBsAg, Troponin I, TSH, Myoglobin, PSA, BNP, and PIVKA-II, the noise levels are greater than about 79,000 conjugate molecules of noise, and are between about 79,000 and 560,000 conjugate molecules of noise. On the right side of the chart, for assays performed by the sample analysis system using digital detection, the noise levels are less than about 1800 conjugate molecules of noise and are between about 300 and 1800 conjugate molecules of noise. Thus, the sample analysis system using digital detection can have a noise reduction of over 99% compared to the sample analysis system using analog detection.

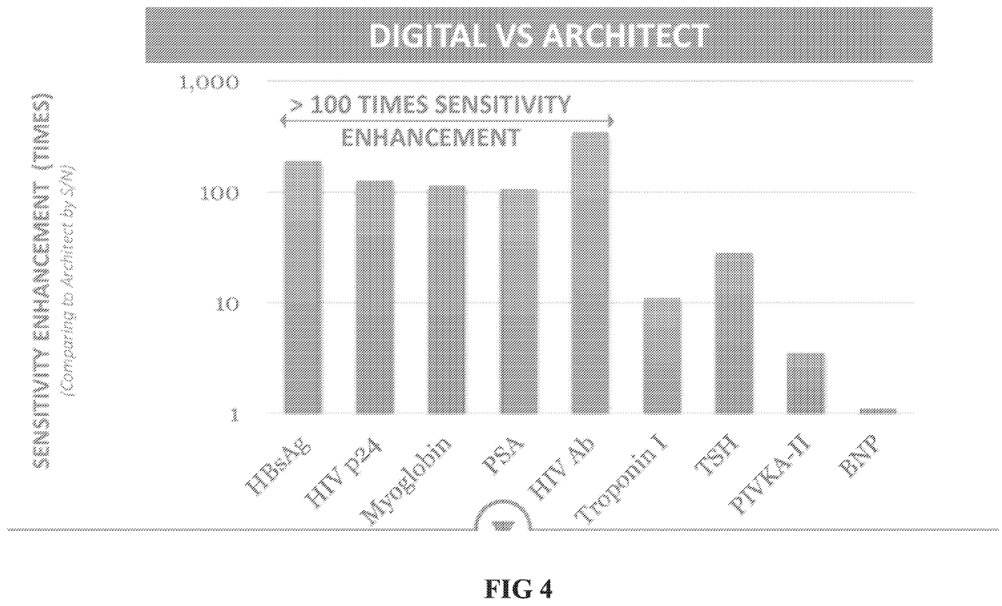

FIG. 4 is a diagram showing the sensitivity enhancement of the sample analysis system using digital detection compared to the sample analysis system using analog detection. For the purpose of illustration and not limitation, for the assays of HBsAg, HIV p24, Myoglobin, PSA, and HIV Ab, the sample analysis system using digital detection has over 100 times sensitivity enhancement compared to the sample analysis system using analog detection. For the assays of Troponin I and TSH, the sample analysis system using digital detection has over 10 times sensitivity enhancement compared to the sample analysis system using analog detection. For the assay of PIVKA-II, the sample analysis system using digital detection has about 5 times sensitivity enhancement compared to the sample analysis system using analog detection.

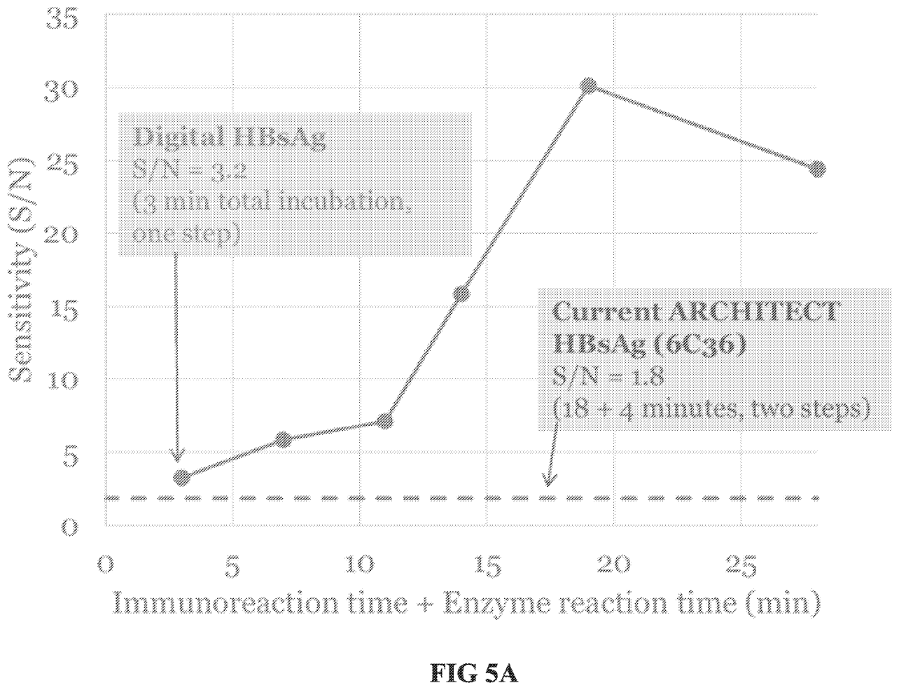

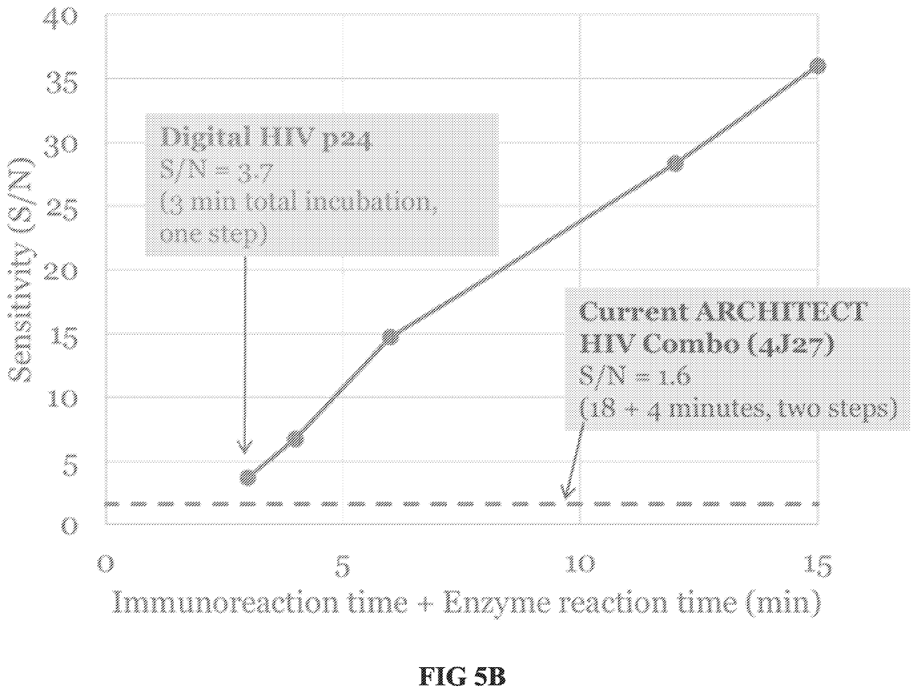

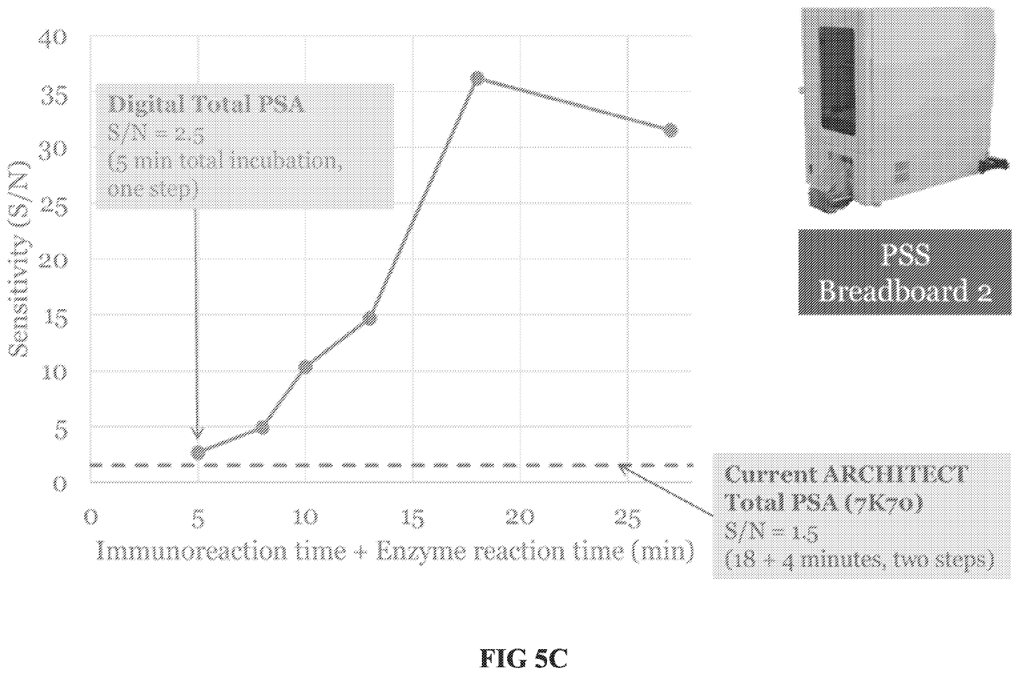

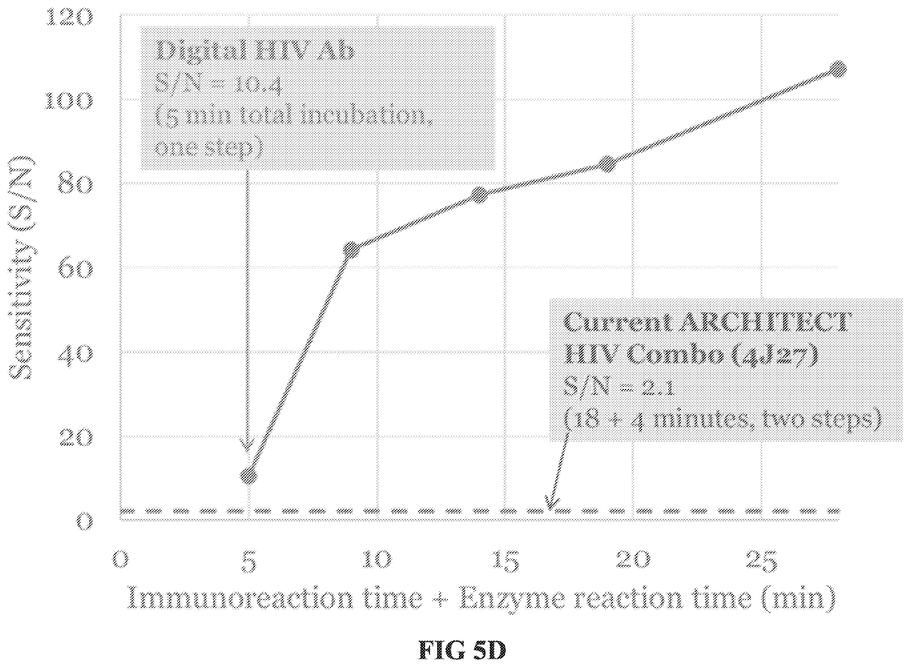

The above data highlights that features of digital detection can be leveraged to improve overall test processing. As described herein, digital detection can be performed using a lower concentration of analyte compared to analog detection, which can allow for decreased time to process the sample to obtain a signal level or concentration suitable for detection. As embodied herein, sample processing can involve a reduced total incubation time, which, for purpose of illustration and not limitation, can be performed as one step, or alternatively, can involve two steps including an immunoreaction time and an enzyme reaction time to obtain the total incubation time. FIG. 5A is a diagram illustrating incubation times to achieve various signal-to-noise (S/N) ratios by an exemplary assay surface using digital detection compared to the sample analysis system using analog detection to perform an HBsAg assay. For example only, and not limitation, the incubation was performed as follows. About 10 μL of sample was applied for the digital HBsAg assay. The X-axis indicates immunoreaction time and enzymatic reaction time. The sensitivity (S/N) was calculated from the signal from the positive sample divided by the signal from the negative sample. The sample volume of the comparable analog detection of HBsAg assay was 75 μL. As shown in FIG. 5A, the assay surface using digital detection can perform the HBsAg assay using one-step incubation with 3 minutes of incubation time to achieve a S/N ratio of 3.2. By comparison, the sample analysis system using analog detection can perform the HBsAg assay using two-step incubation with an immunoreaction time of 18 minutes and an enzyme reaction time of 4 minutes for a total incubation time of 22 minutes to achieve a S/N ratio of 1.8. As such, the assay surface using digital detection can achieve about a 75% increase in sensitivity in about one-eighth (⅛) the time for incubation for the HBsAg assay compared to the sample analysis system using analog detection.

FIG. 5B is a diagram illustrating incubation times to achieve various S/N ratios by the exemplary assay surface using digital detection compared to the sample analysis system using analog detection to perform an HIV p24 assay. For example only, and not limitation, the incubation was performed as follows. About 10 μL of sample was applied for the digital HIV p24 assay. The X-axis indicates immunoreaction time and enzymatic reaction time. The sensitivity (S/N) was calculated from the signal from the positive sample divided by the signal from the negative sample. The sample volume of the comparable analog detection of HIV Ag/Ab Combo assay was 100 μL. As shown in FIG. 5B, the assay surface using digital detection can perform the HIV p24 assay using one-step incubation with 3 minutes of incubation time to achieve a S/N ratio of 3.7. By comparison, the sample analysis system using analog detection can perform the HIV p24 assay using two-step incubation with an immunoreaction time of 18 minutes and an enzyme reaction time of 4 minutes for a total incubation time of 22 minutes to achieve a S/N ratio of 1.6. As such, the assay surface using digital detection can achieve about a 130% increase in sensitivity in about one-eighth (⅛) the time for incubation for the HIV p24 assay compared to the sample analysis system using analog detection.

FIG. 5C is a diagram illustrating incubation times to achieve various S/N ratios by the assay surface using digital detection compared to the sample analysis system using analog detection to perform a PSA assay. For example only, and not limitation, the incubation was performed as follows. About 10 μL of sample was applied for the digital total PSA assay. The X-axis indicates immunoreaction time and enzymatic reaction time. The sensitivity (S/N) was calculated from the signal from the positive sample divided by the signal from the negative sample. The sample volume of the comparable analog detection of total PSA assay was 50 μL. As shown in FIG. 5C, the assay surface using digital detection can perform the PSA assay using one-step incubation with 5 minutes of incubation time to achieve a S/N ratio of 2.5. By comparison, the sample analysis system using analog detection can perform the PSA assay using two-step incubation with an immunoreaction time of 18 minutes and an enzyme reaction time of 4 minutes for a total incubation time of 22 minutes to achieve a S/N ratio of 1.5. As such, the assay surface using digital detection can achieve about a 67% increase in sensitivity in about one-fourth (¼) the time for incubation for the PSA assay compared to the sample analysis system using analog detection.

FIG. 5D is a diagram illustrating incubation times to achieve various S/N ratios by the assay surface using digital detection compared to the sample analysis system using analog detection to perform an HIV Ab assay. For example only, and not limitation, the incubation was performed as follows. About 10 μL of sample was applied for the digital HIV Ab assay. The X-axis indicates immunoreaction time and enzymatic reaction time. The sensitivity (S/N) was calculated from the signal from the positive sample divided by the signal from the negative sample. The sample volume of the comparable analog detection of HIV Ag/Ab Combo assay was 100 μL. As shown in FIG. 5D, the assay surface using digital detection can perform the HIV Ab assay using one-step incubation with 5 minutes of incubation time to achieve a S/N ratio of 10.4. By comparison, the sample analysis system using analog detection can perform the HIV Ab assay using two-step incubation with an immunoreaction time of 18 minutes and an enzyme reaction time of 4 minutes for a total incubation time of 22 minutes to achieve a S/N ratio of 2.1. As such, the assay surface using digital detection can achieve about a 500% increase in sensitivity in about one-fourth (¼) the time for incubation for the HIV Ab assay compared to the sample analysis system using analog detection.

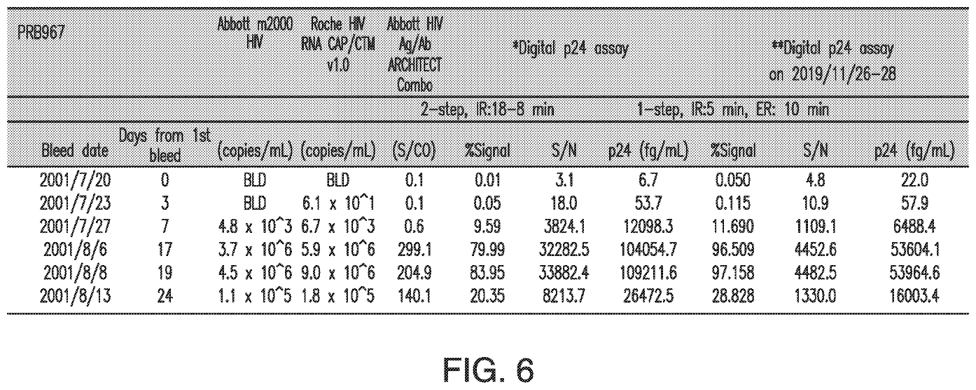

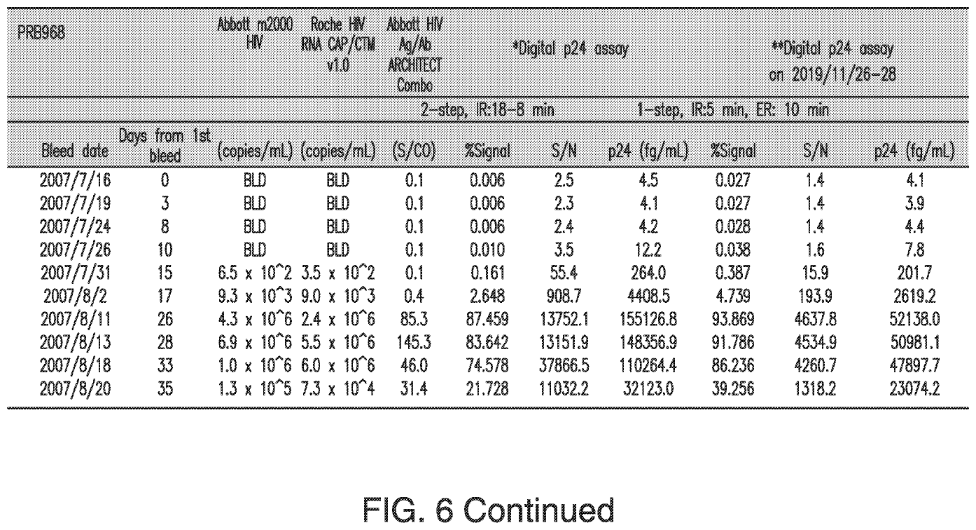

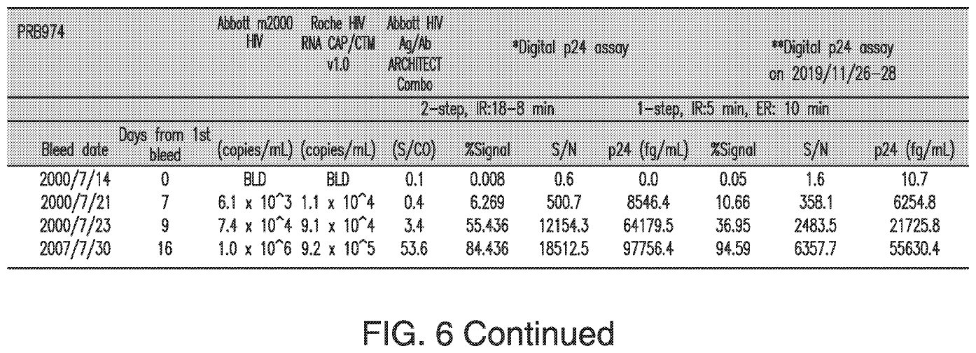

FIG. 6 is a chart illustrating improved sensitivity based on additional data obtained from a seroconversion panel evaluation of an HIV p24 assay by assay surfaces using digital detection compared to the sample analysis systems using analog detection (e.g., Abbott m2000 HIV, Roche HIV RNA CAP/CTM v.1.0, and Abbott HIV Ag/Ab ARCHITECH™ systems). As shown in FIG. 6, assay surfaces using digital detection have improved sensitivity compared to the sample analysis systems using analog detection.

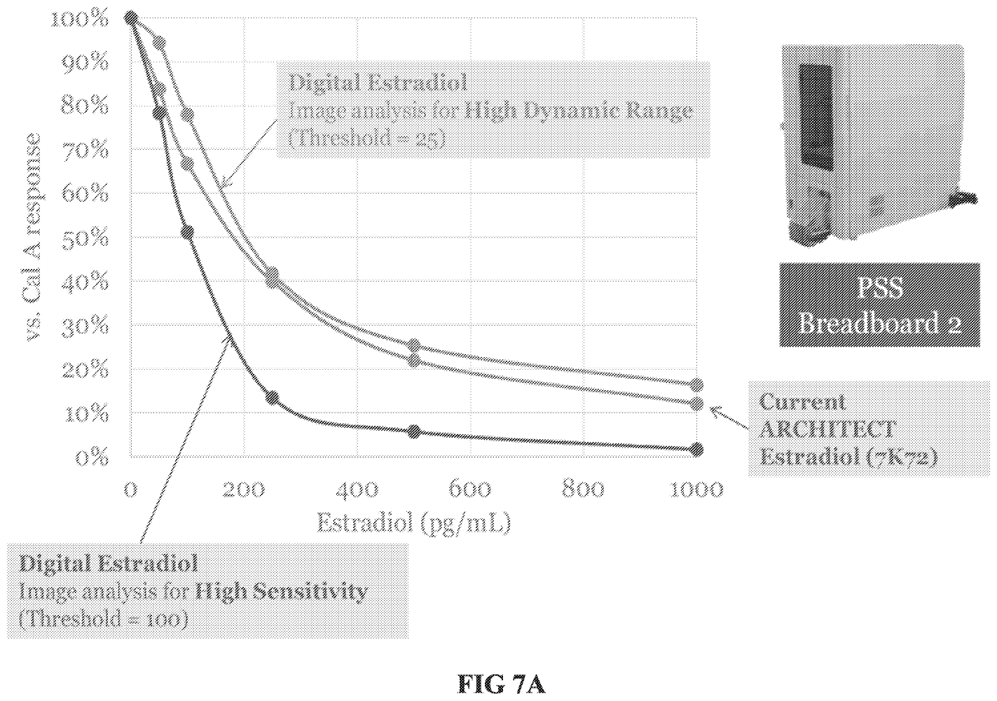

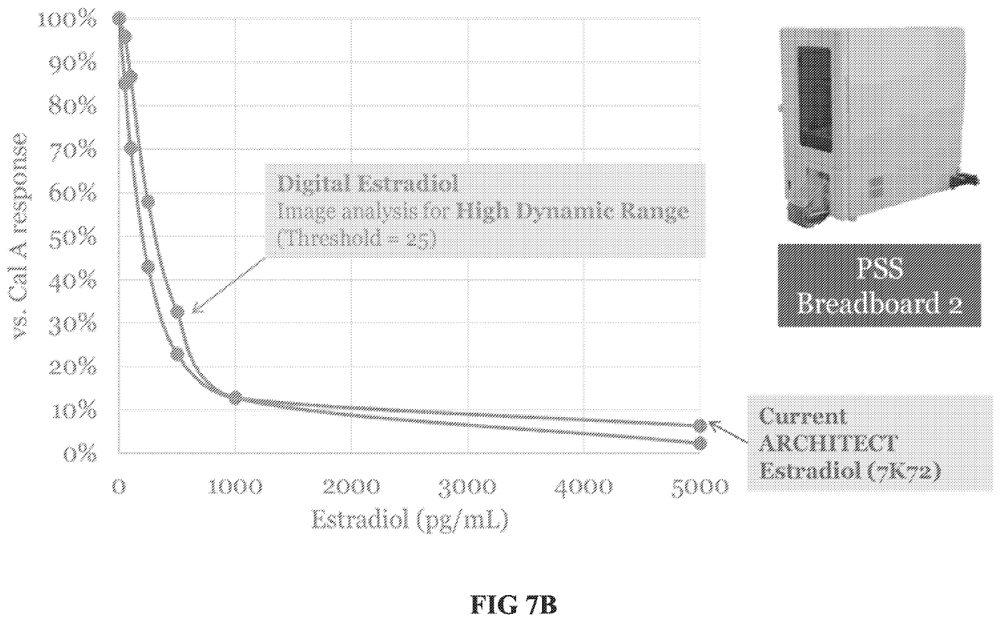

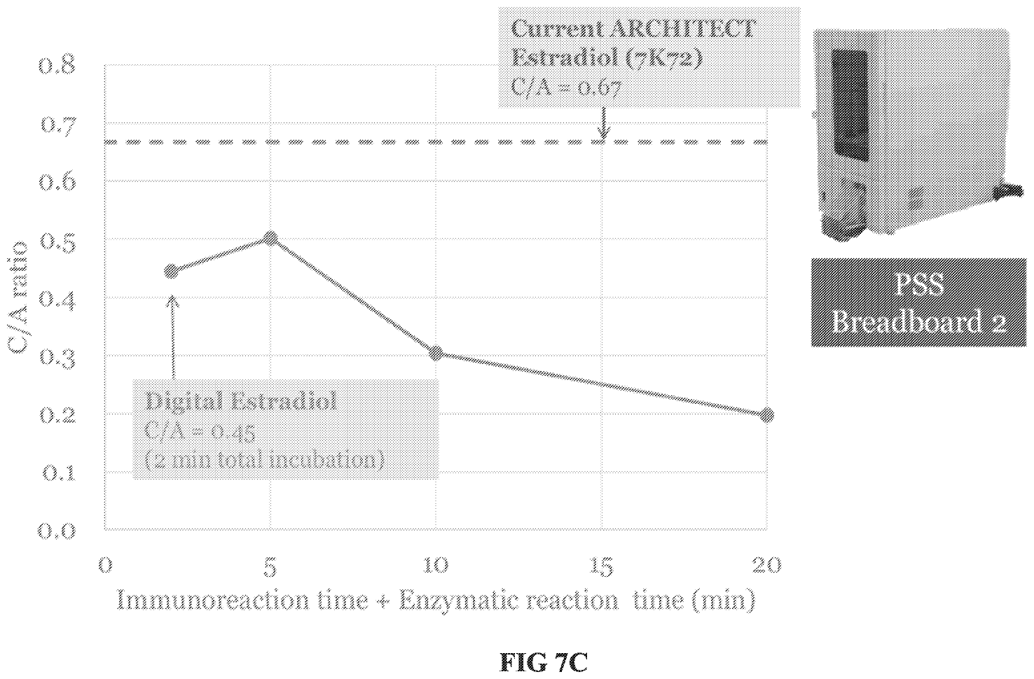

Digital detection can be configured to provide increased dynamic range of detection, in addition or as an alternative to increased sensitivity, compared to the sample analysis systems using analog detection. FIGS. 7A-7B is a diagram illustrating exemplary calibration curves for an Estradiol assay by an exemplary assay surface using digital detection configured for high sensitivity and an assay surface using digital detection configured for high dynamic range compared to a sample analysis system using analog detection. As shown in FIG. 7A, the curve labeled “High Sensitivity” illustrates an image analysis configured for high sensitivity having a threshold of 100 units of reactive intensity measured by the detector for the digital detection of estradiol. As shown in FIGS. 7A-7B, the curve labeled “High Dynamic Range” illustrates an image analysis configured for high dynamic range having a threshold of 25 units of reactive intensity measured by the detector for the digital detection of estradiol. By comparison, in FIGS. 7A-7B, the curve labeled “ARCHITECT™” illustrates an image analysis by a sample analysis system using analog detection (e.g., Abbott ARCHITECT™). As shown in FIG. 7A, compared to ARCHITECT™, the High Sensitivity digital configuration has a greater response at lower concentrations of estradiol. As shown in FIGS. 7A-7B, compared to ARCHITECT™, the High Dynamic Range digital configuration has a greater response at higher concentrations of estradiol. As such, compared to sample analysis systems using analog detection, assay surface using digital detection can be configured to have a similar sensitivity with a higher dynamic range, or higher sensitivity with a similar dynamic range, or a combination of higher sensitivity and higher dynamic range.

FIG. 7C is a diagram illustrating exemplary calibration curves for a competition assay of Estradiol by an assay surface using digital detection compared to a sample analysis system using analog detection. The vertical axis shows the Cal C signal per noise (C/A ratio), which indicates sensitivity for the estradiol assay, where a lower C/A ratio indicates a higher sensitivity. As shown in FIG. 7C, after 2 minutes of incubation time, the assay surface using digital detection has a C/A ratio of 0.45, which is lower than the sample analysis system using analog detection having a C/A ratio of 0.67.

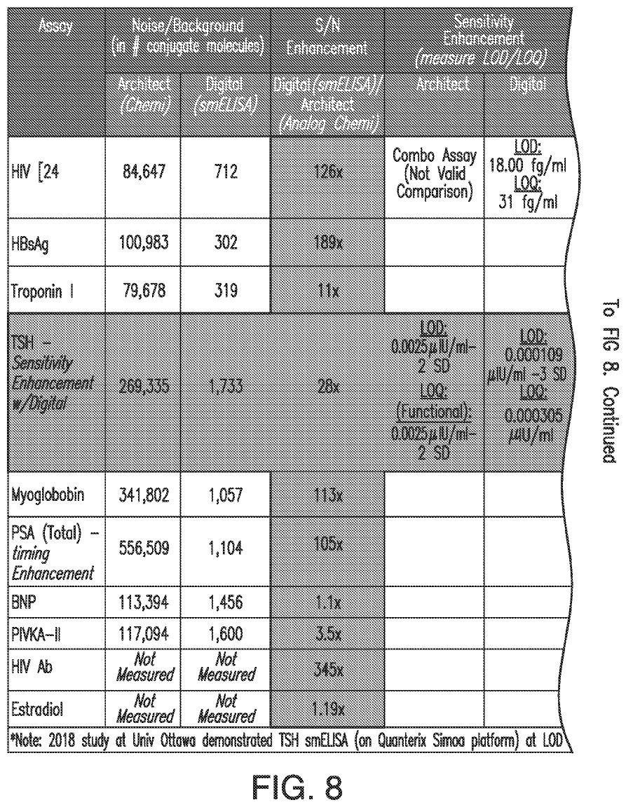

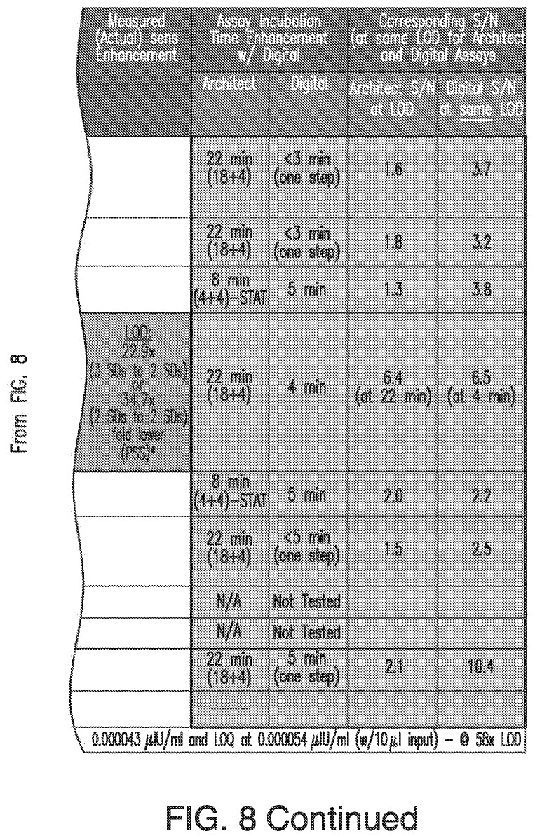

FIG. 8 shows data for various assays performed by an exemplary assay surface using digital detection compared to sample analysis systems using analog detection (e.g., Abbott ARCHITECT™) for purpose of illustration and confirmation of the disclosed subject matter. For example and without limitation, TSH assays were performed. As shown in FIG. 8, S/N ratio of assay surfaces using digital detection was 28 times higher than sample analysis systems using analog detection for the TSH assay. The limit of detection (LOD) of sample analysis systems using digital detection was at least 22.9 times lower than sample analysis systems using analog detection for the TSH assay.

To obtain a similar limit of detection (LOD) with a similar S/N ratio, assay surfaces using digital detection utilized 4 minutes of incubation time compared to sample analysis systems using analog detection that utilized 22 minutes of incubation time. As such, the digital detection systems describe herein allow for significantly shorter sample processing time than required to achieve a suitable result for analog detection. As shown in FIG. 8, compared to analog detection, for other comparable assays, the assay surfaces using digital detection have comparable or higher sensitivity and shorter processing time compared to sample analysis systems using analog detection. For example, for those assays tested and measured shown in FIG. 8, assay surfaces using digital detection enhanced the detection sensitivity based on S/N ratio from 11 times to 189 times.

According to other aspects of the disclosed subject matter, assay surfaces using digital detection can be configured to have higher dynamic range of detection, in addition or as an alternative to higher sensitivity, compared to sample analysis systems using analog detection alone. When concentration of an analyte of interest in a sample exceeds a threshold, the detection component can become saturated such that further increase in concentration does not produce a measurable change in the signal detectable by the detection component.

Configurations to increase dynamic range by assay surfaces using digital detection can result in various improvements in the assay, including cost and time improvements. For example, various conditions of the assay can be modified to take advantage of increased dynamic range. For the purpose of illustration and not limitation, modifications to the assay conditions can include reducing the volume of the sample, increasing the substrate concentration in the sample, decreasing the microparticle concentration or conjugate concentration in the sample, or any combination of such modifications or similar modifications.

Additionally or alternatively, configurations of the sample analysis system can be modified to take advantage of increased dynamic range. For the purpose of illustration and not limitation, the sample analysis system can be modified to shorten the enzyme reaction time before detection or use rates for more precise control of the enzyme reaction signal, or any combination of such modification or similar modifications.

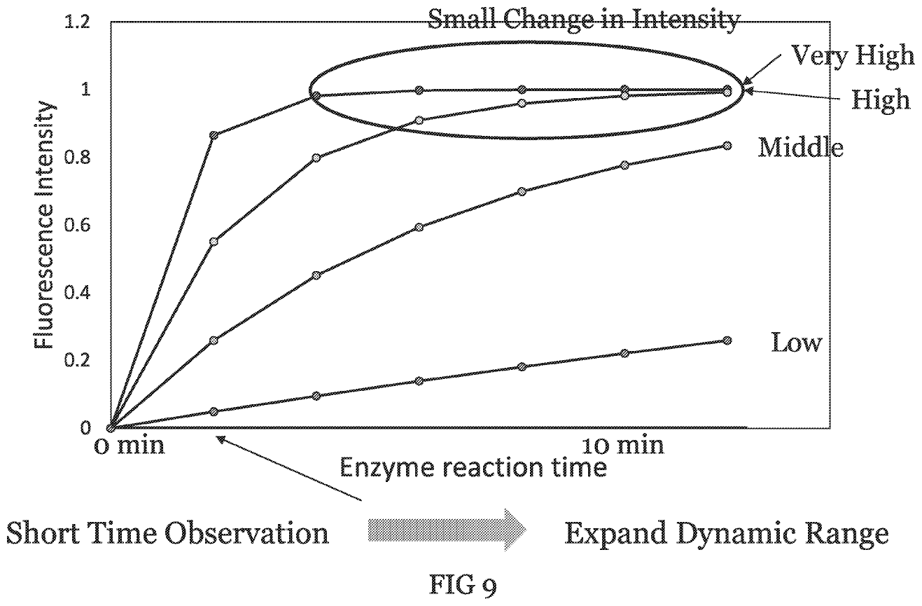

FIG. 9 is a diagram illustrating changes in fluorescence intensity over enzyme reaction time for exemplary assays. As shown in FIG. 9, during certain high-concentration assays, when the enzyme reaction increases, there can be only a small or no change in the detection signal, which can be due to saturation. As such, when detection of a sample occurs after a certain amount of incubation, the duration of which can vary depending on the type and conditions of the assay, the fluorescence signals do not provide a measurable difference in intensity as concentration increases, at which point the detection system can be considered saturated. Thus, shortening the observation time in a sample analysis system can allow for differences in intensity to be measured for a wider range of concentrations in an expanded dynamic range, and images can be taken at any one or more points during the enzyme reaction time to obtain one or more intensities corresponding to a concentration of an analyte of interest in the sample.

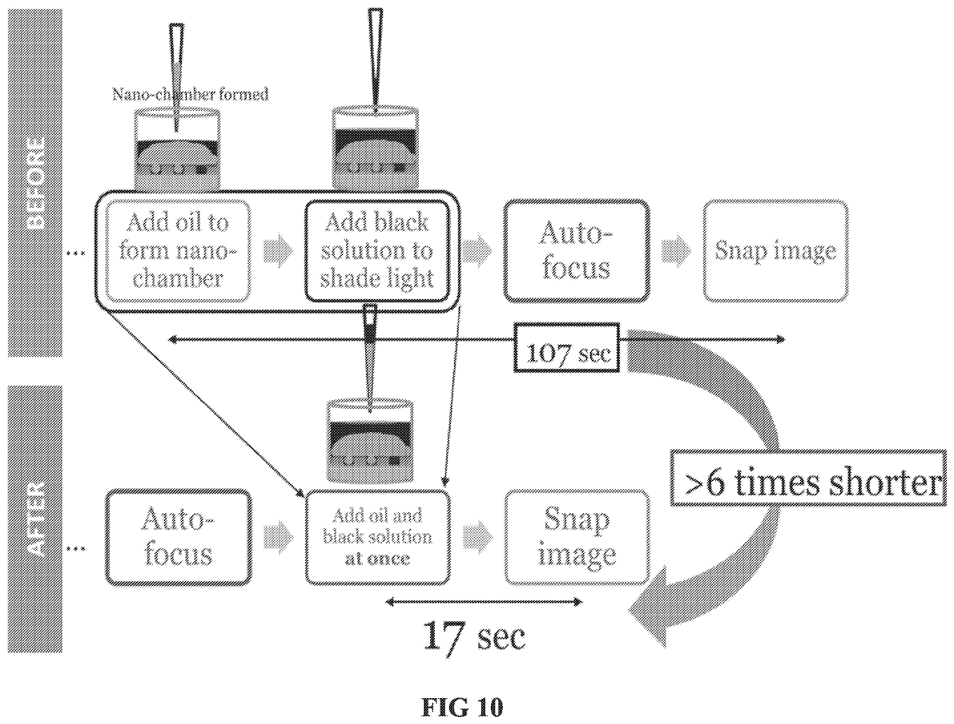

FIG. 10 shows an exemplary modification to an assay surface using digital detection to shorten the observation time. For the purpose of illustration and not limitation, with reference to FIG. 10, an exemplary detection method (1000) is illustrated. At (1001), oil is added to an analyte solution to form nano-chambers for detection. At (1002), black dye is added to the analyte solution to shade the background and increase contrast for optical detection. At (1003), an optical detection device (e.g., a CCD camera) is focused to resolve the image of the analyte solution, and at (1004), the optical detection device obtains an image of the analyte solution for detection. From the oil addition (1001) to image capture (1004), the time to perform detection method (1000) is about 107 seconds.

Referring still to FIG. 10, for the purpose of illustration and not limitation, an exemplary detection method (1010) according to the disclosed subject matter is illustrated. At (1011), an optical detection device (e.g., a CCD camera) is focused to resolve the image of the analyte solution. At (1012), both oil and black solution are added at once to the analyte solution. At (1013), the optical detection device obtains an image of the analyte solution for detection. The time to perform detection method (1010) is about 17 seconds, which is about 6 times shorter than detection method (1000). By shortening the observation time window, the dynamic range can be increased, as described herein.

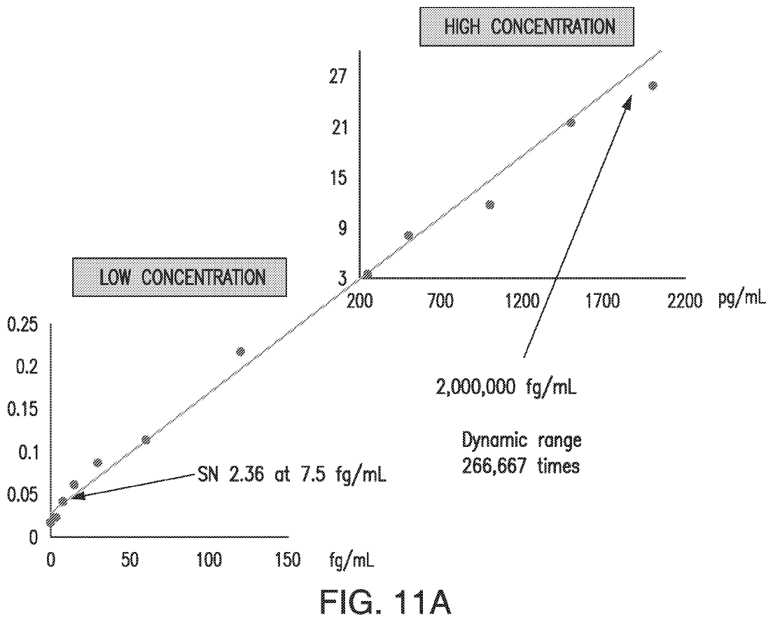

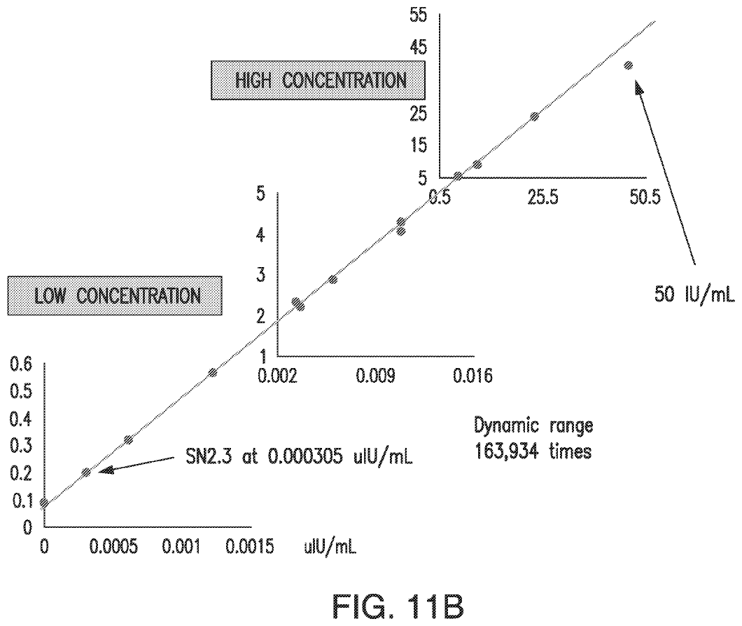

FIG. 11A illustrates additional details of the expanded dynamic range of an assay surface using digital detection according to the disclosed subject matter for an HIV p24 assay. As shown in FIG. 11A, the assay surface using digital detection is responsive to both low concentrations and high concentration of analyte in the HIV p24 assay, for example, as shown from about 7.5 fg/mL to up to 2000 μg/mL, for a dynamic range of about 266,667 times (e.g., 2000 μg/mL divided by 7.5 fg/mL). For purpose of illustration and comparison with the disclosed subject matter, and not limitation, the assay range of the ARCHITECT™ HIV p24 assay is about 5,000-10,000 times dynamic range. In conventional systems, dynamic range can be extended, for example and without limitation, by taking a first image at a higher concentration, diluting the sample, and taking a second image at a lower concentration. However, such dilution processes can involve additional processing time and steps to extend dynamic range.

FIG. 11B illustrates additional details of the expanded dynamic range of an assay surface using digital detection according to the disclosed subject matter for an TSH assay. As shown in FIG. 11B, the assay surface using digital detection is responsive to both low concentrations and high concentration of analyte in the TSH assay, for example, as shown from about 0.000305 μIU/mL to up to 50 μIU/mL, for a dynamic range of about 163,934 times (e.g., 50 μIU/mL divided by 0.000305 μIU/mL). For purpose of illustration and comparison with the disclosed subject matter, and not limitation, the assay range of the ARCHITECT™ TSH assay is from 0.01 μIU/mL to 100 μIU/mL (e.g., about 10,000 times dynamic range), which can be extended up to about 500 μIU/mL for example and without limitation by a dilution process.

According to other aspects of the disclosed subject matter, exemplary assay surfaces for use with exemplary assay processing units (APUs), assay processing systems (APSs), and laboratory systems are provided. Systems and methods for sample analysis can use any suitable components and techniques for sample processing and detection. For example and without limitation, for all or part of sample processing and detection, a pipette or system of pipettes can be used to perform washing, mixing or any other steps to form, isolate, purify or otherwise manipulate an analyte solution, to incubate or combine the analyte solution with reaction components, and/or to move the analyte solution to a detection location.

Additionally or alternatively, all or part of sample processing and/or detection can be performed using various reaction vessels and automated processing, including automated pipette systems using suction or vacuum forces to manipulate analyte solutions, or other automated systems using other forces, such as magnetic forces or dielectrophoresis, to manipulate analyte solutions.

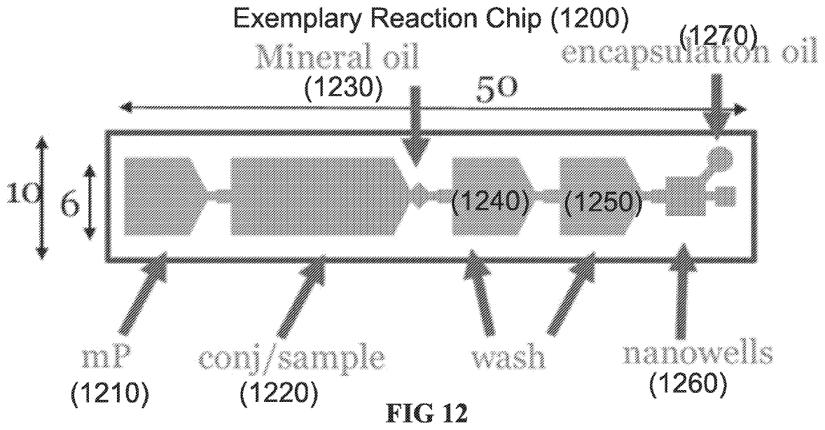

For purpose of illustration and not limitation, referring now to FIG. 12, as embodied herein, an exemplary assay surface (1200) can be used in sample analysis systems according to the disclosed subject matter to perform all or part of sample processing and/or moving the analyte to a region for detection within an added magnetic field. For purpose of illustration not limitation, as embodied herein, assay surface (1200) using magnetic force described herein can include a reaction chip made of hydrophobic material. Alternatively, assay surfaces according to the disclosed subject matter can other suitable surfaces for sample preparation and detection. Assay surface (1200) can be configured as a series of regions through which microparticles can be moved by translation of a moving magnetic field, for example, a moving magnet or an electromagnet, parallel to the microparticles to perform various operations as described herein. Each region can be separated by a barrier or other separation mechanism, which can be an air-to-liquid interface, an liquid-to-immiscible liquid interface (for example, separating an oil region from another liquid region), a valve, a plurality of stopping elements, or any other suitable separation mechanism.

For example and without limitation, assay surface (1200) includes a microparticle (mP or μP) storage region (1210) configured to hold one or more microparticles (or beads). As embodied herein, the microparticles (or beads) can already be stored in the storage region (1210). Alternatively, microparticles (or beads) can be added to the assay surface manually or by automatically pipetting system from a larger reservoir of microparticles. As described herein, the microparticles (or beads) can be magnetic or paramagnetic to facilitate the use of magnet forces to perform sample analysis. Microparticle storage region (1210) can be configured as a flat surface or can have a volume sized to hold a suitable number of microparticles to perform the sample analysis.

Assay surface (1200) can include a sample/conjugate mixing region (1220) extending from microparticle storage region (1210). As embodied herein, the sample/conjugate mixing region (1210) can include pre-loaded reagents or conjugates. Additionally or alternatively, reagents or conjugates can be added to the assay surface manually or by automatically pipetting system from a larger reservoir. Sample/conjugate mixing region (1220) can include or be configured to receive one or more analytes of interest to bind to one or more microparticles moved into the sample/conjugate mixing region (1220). For example and without limitation, samples can be stored on the assay surface, or can be moved to the sample/conjugate mixing region by manual or automatic pipetting or any other suitable technique. Sample/conjugate mixing region (1220) can be configured as a flat surface or can have a volume sized to hold a suitable number of samples, conjugates, enzymes, or other reagents for use by the assay surface to detect an analyte of interest in the sample.

Assay surface (1200) can include one or more liquid volumes. For example and without limitation, assay surface (1200) can include an inert fluid region (1230) extending from sample/conjugate mixing region (1220). As embodied herein, inert fluid region (1230) can include, for example, a mineral oil, or other inert fluid immiscible with the sample, which can facilitate formation of sample droplets as well as increase stability of the shape of sample droplets and can further be useful for keeping sample droplets and microparticles spatially separated from one another. Additionally or alternatively, and as embodied herein, inert fluid region (1230) can be configured to perform a washing function, for example and without limitation, to remove excess aqueous solution from the microparticles when passed through the mineral oil. Additional or alternative washing steps can be performed to remove other contaminants as described herein. As embodied herein, the mineral oil in inert fluid region (1230) can be any mineral oil suitable (e.g., Nacalai Tesque Code 23306-84). Mineral oil can include a mixture of liquid hydrocarbons and can be derived from crude oil by distillation and refining. Other suitable oils for use in inert fluid region (1230) can include Fluorine oils (e.g., FC-40) and organic oils (e.g., grapeseed oil, coconut oil, or Theobroma oil).

Assay surface (1200) can also include one or more additional wash regions (1240, 1250), for example and without limitation, extending from, or instead of, inert fluid region (1230). Wash regions (1240, 1250) each can define a liquid volume with an air-to-aqueous interface at each end thereof. The wash regions can include a solution, such as a buffer solution or any suitable solution to remove unwanted contaminants or excess materials, such as excess reagents or conjugates not bound to an analyte of interest or any microparticles or beads. Surface tension can be applied to the microparticles as the microparticles move through the air-to-aqueous interfaces of the wash regions (1240, 1250) to remove unwanted contaminants or excess materials.

Assay surface (1200) can include a detection region (1260) extending from wash regions (1240, 1250). For purpose of illustration and not limitation, as embodied herein, detection region (1260) can include an array of elements, each dimensioned to hold at least a single one of the microparticles or beads. For purpose of illustration not limitation, the array of elements can include an array of nanowells. Each nanowell can be sized to receive a single microparticle for single-molecule detection. Alternatively, the detection region (1260) can be configured as a flat surface.

Assay surface (1200) can include one or more additional regions extending from the detection region (1260). For example, and as embodied herein, end region (1270) can include an encapsulation inert liquid region to store encapsulation inert liquid, for example, oil, for use to encapsulate the detection region (1260). End region (1270) can also include a dye region to store dye to shade the background and increase contrast for detection and, in one embodiment, can be premixed with the oil. End region (1270) can further include a disposal region to move microparticles or any other used components from the assay surface (1200) for disposal.

For purpose of illustration but not limitation, as embodied herein, assay surface (1200) can have a length of about 50 mm with a width of about 10 mm. Each region can have a width up to about 6 mm, for example and as embodied herein. The exemplary assay surface (1200) can be used as part of an assay processing system (APS) with an assay processing unit (APU) in a laboratory system in accordance with the disclosed subject matter.

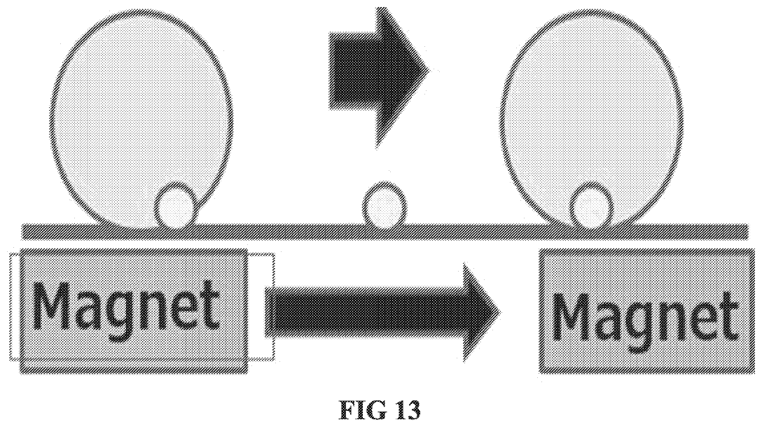

FIG. 13 illustrates exemplary movement of a microparticle along an assay surface (1200) through liquid volumes corresponding to regions. It will be apparent to those skilled in the art that various modifications and variations can be made in the methods and systems disclosed herein. As described herein, at least one moving magnetic field (1301) can be provided to urge the microparticle (1305) along the assay surface through volumes of liquid in different regions and into a detection component of the assay surface. The moving magnetic field (1301) can be generated by a magnetic element disposed in any suitable positions relative to the assay surface. For the purpose of illustration, the magnetic element can be disposed above the assay surface, under the assay surface, on a side of the assay surface, or other suitable locations. For purpose of illustration not limitation, as embodied herein, the at least one moving magnetic field (1301) can be a moving magnet. Alternatively, the moving magnetic field (1301) can be generated by, for example, an electromagnet. For purpose of illustration not limitation, as embodied herein, the moving magnetic field (1301) is disposed under the assay surface. Alternatively, the moving magnetic field (1301) can be disposed in other suitable positions. As embodied herein, some or all of the surface of assay surface (1200) can be made of a hydrophobic material, which can prevent or inhibit unwanted movement of liquid between the regions. Surface tension can be applied to the microparticle as the microparticle moves, for example and without limitation, through the air-to-liquid interfaces or air-to-oil interfaces of the various regions from sample processing to detection.

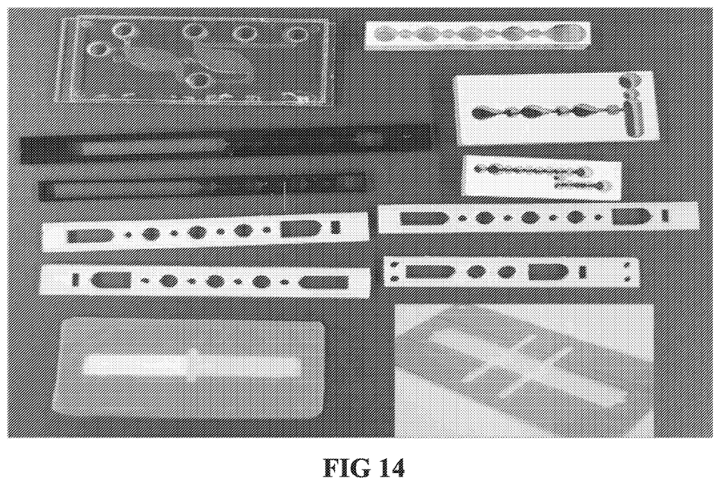

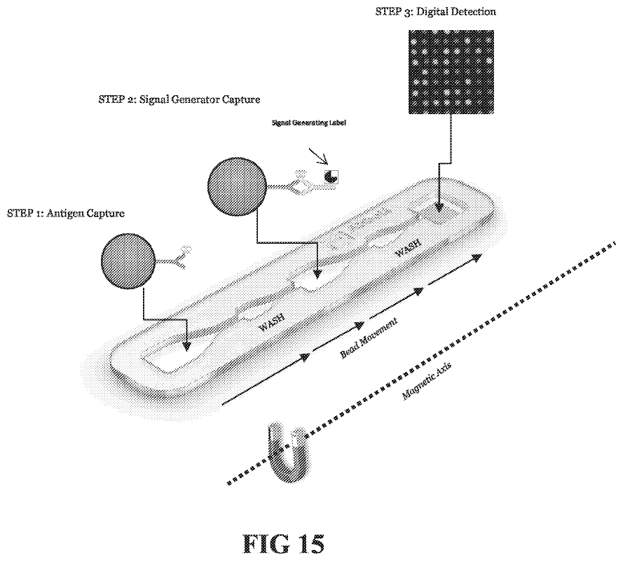

FIGS. 14-15 illustrate alternative embodiments of assay surfaces having different configurations according to the disclosed subject matter. With reference to FIG. 15, an assay surface (1500) can include five regions. Microparticles or beads are moved through each region of assay surface (1500) and into a detection region (1550) along the length of assay surface (1500) by magnetic force. For purpose of illustration only, a magnetic axis is depicted in FIG. 15 as below the assay surface (1500). Alternatively, the magnetic force can be generated by a magnetic element at other suitable locations, for example, above the assay surface (1500), or by a side of the assay surface (1500).

As shown in FIG. 15, as embodied herein, assay surface (1500) can include a sample region (1510). Sample region (1510) includes microparticles to be combined with a sample having an analyte (antigen) of interest, for example, by pipetting or any other suitable technique. Alternatively, sample region (1510) can be pre-loaded with microparticles. In the sample region (1510), for example, a microparticle can be bound to a single antigen in the sample as the first binding partner.

Assay surface (1500) can include a wash region (1520) extending from sample region (1510). As described herein, a single wash region (1520) is embodied, however, additional wash regions can also be included. Wash region (1520) can be configured to remove unwanted contaminants and/or unbound analytes from the microparticle, as described herein.

Assay surface (1500) can include a conjugate/enzyme region (1530) extending from wash region (1520). For purpose of illustration, region (1530) can include reagents or conjugates, or alternatively, reagents or conjugates can be added to the region manually or automatically using, for example, a pipettor. In the conjugate/enzyme region (1530), the analytes (antigens) bound to microparticles can bind with another analyte-specific binding partner as the second binding partner, labelled to produce a signal for detection.

Assay surface (1500) can include a wash region (1540) extending from conjugate/enzyme region (1530). As described herein, a single wash region (1540) is embodied, however, additional wash regions can also be included. Wash region (1540) can be configured to remove unbound conjugates/reagents, as described herein.

Assay surface (1500) can include a detection region (1550) extending from wash region (1540). As embodied herein, detection region (1550) can be configured as a digital detection region. Alternatively, detection region (1550) can be configured to perform other suitable detections, for example, analog detection. Detection region (1550) can include one or more nanowells configured for detection. Alternatively, digital detection region can be configured as a flat surface. Additionally or alternatively, for purpose of illustration but not limitation, the detection region (1550) can include other area where the microparticles are detected and/or imaged, including using nanowells, nanopores, fluorescent detection areas, or any other suitable region for detection of analytes in an assay. Exemplary assay surfaces described herein can be formed from any suitable materials, for example and without limitation, from a PTFE sheet or any other suitable material (e.g., cyclic olefin polymer (COP), PMMA, or other hydrophobic material).

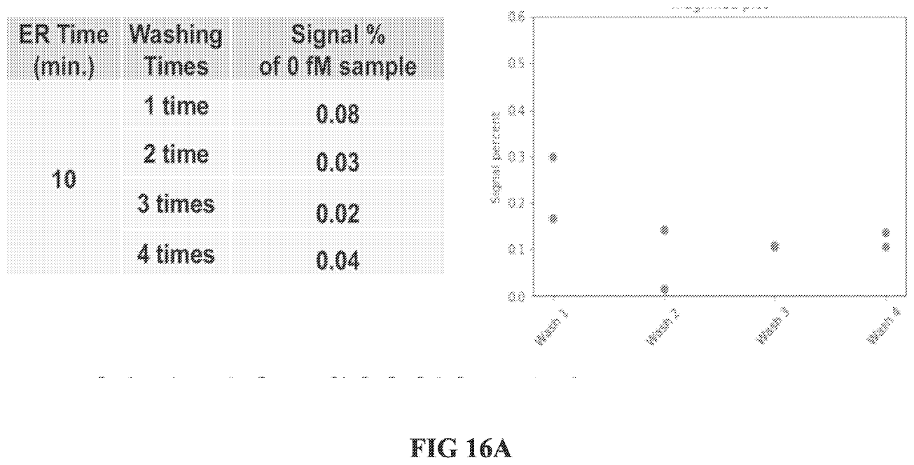

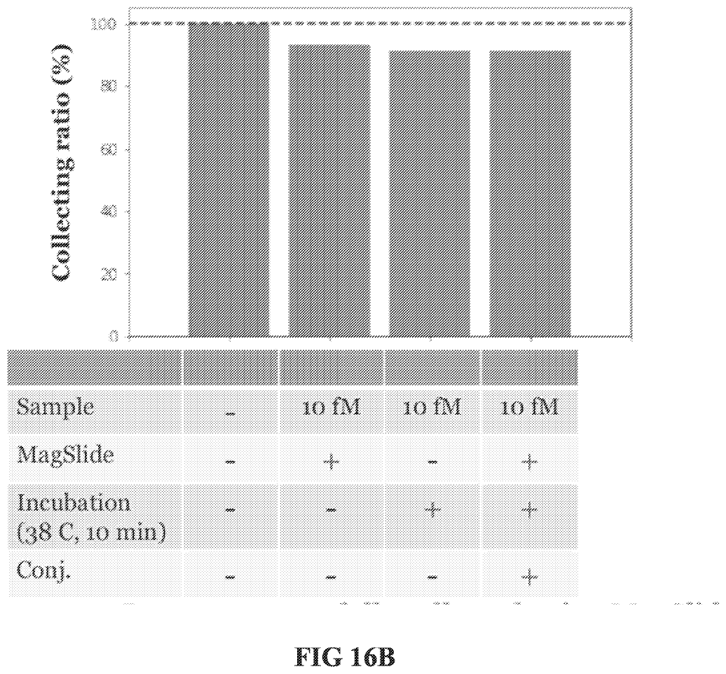

Exemplary assay surfaces described herein can be used to perform sample processing, including, for example and without limitation, any sample processing steps described herein. FIG. 16A illustrates exemplary washing efficiency for an HBsAg assay performed using assay surfaces according to the disclosed subject matter. For example and without limitation, 75 μL of negative sample (recalcified plasma) and HBsAg assay beads were incubated for 18 minutes. After incubation, beads were attracted and moved on a hydrophobic surface by a moving magnetic field. The wash processes were conducted by passing the collected beads using the magnetic field through 10 μL buffer droplets. Up to 4 washes were performed during the assay. As shown in FIG. 16A, after a first wash, a signal percentage of 0.08 was obtained. After a second wash, a signal percentage of 0.03 was obtained, which can be suitable for digital detection as described herein. The signal percentage can be considered as a percentage of beads with bright droplets counted from the total number of collected beads, and for example and without limitation, can be determined by the following equation: NbD/NtB×100%, where NbD and NtB refer to the number of beads with bright droplets and the total number of collected beads, respectively. Additional washes produced a smaller change in signal percentage obtained. As such, two washes can be suitable to perform assays using assay surfaces according to the disclosed subject matter, and a total wash time can be about 30 seconds.

FIG. 16B illustrates exemplary collecting efficiency using assay surfaces according to the disclosed subject matter. As shown in FIG. 16B, assay surfaces according to the disclosed subject matter can have a greater than 90% collecting efficiency ratio (e.g., a number of microparticles remaining after an assay) illustrating suitable collection of microparticles using assay surfaces disclosed herein. With reference FIG. 16B, the column labeled “−/−/−/−” indicates the initially untreated beads (e.g., 100% collecting ratio). The column labeled “10 fM/+/−/−” indicates the collecting ratio of beads with a 75 μL sample assay surfaces according to the disclosed subject matter (e.g., greater than 90% collecting ratio). The column labeled “10 fM/−/+/− indicates the collecting ratio of beads with 75 μL sample without assay surfaces according to the disclosed subject matter and without conjugate (e.g., about 90% collecting ratio). The column labeled “10 fM/+/+/+” indicates the collecting ratio of beads with HBsAg assay with a 75 μL sample using assay surfaces and with incubation and conjugate added according to the disclosed subject matter (e.g., about 90% collecting ratio). As such, a high percentage of microparticles are retained by the assay surfaces according to the disclosed subject matter as the microparticles are moved along the various regions of the assay surfaces.

As discussed herein, detection according to the disclosed subject matter can be performed using a smaller sample volume, less reagent material and volume, less conjugate material, fewer nanoparticles, or any combination of these, which can reduce costs to perform each assay. As such, sample preparation time can be improved due at least in part to less sample manipulation involved. Smaller sample volumes can also provide certain kinetics improvements to improve sample processing speed, for example during incubation or amplification reactions or other reactions performed using such sample volumes. As embodied herein, sample analysis systems using exemplary assay surfaces according to the disclosed subject matter can be configured to improve processing time of smaller volumes of samples, conjugates and/or microparticles.

Sample processing systems and techniques described herein can be used to perform sample processing of small sample volumes, for example and without limitation about 10 μL or less. Alternatively, the sample volume for exemplary assay surfaces can be between about 10 μL and about 50 μL. Alternatively, the sample volume for exemplary assay surfaces can be less than 50 μL. Alternatively, the sample volume for exemplary assay surfaces can be less than 75 μL. Alternatively, the sample volume for exemplary assay surfaces can be less than 100 μL. Additionally or alternatively, exemplary assay surfaces according to the disclosed subject matter can provide faster washing times, including when used with small sample volumes. By comparison, some conventional sample analysis systems can be unsuitable for use with sample volumes less than 100 μL.

Additionally or alternatively, sample processing systems and techniques described herein can be used to perform sample processing using small wash buffer volumes, for example and without limitation about 10 μL or less. Alternatively, the wash buffer volume for exemplary assay surfaces can be between about 10 μL and about 50 μL. Alternatively, the wash buffer volume for exemplary assay surfaces can be less than 50 μL. Alternatively, the wash buffer volume for exemplary assay surfaces can be less than 75 μL. Alternatively, the wash buffer volume for exemplary assay surfaces can be less than 100 μL. Additionally or alternatively, exemplary assay surfaces according to the disclosed subject matter can provide faster washing times, including when used with small sample volumes. By comparison, some conventional sample analysis systems can be unsuitable for use with wash buffer volumes less than 100 μL.

Additionally or alternatively, sample processing systems and techniques described herein can be used to perform sample processing using small reagent volumes, for example and without limitation about 10 μL or less. Alternatively, the reagent volume for exemplary assay surfaces can be between about 10 μL and about 50 μL. Alternatively, the reagent volume for exemplary assay surfaces can be less than 50 μL. Alternatively, the reagent volume for exemplary assay surfaces can be less than 75 μL. Alternatively, the reagent volume for exemplary assay surfaces can be less than 100 μL. Additionally or alternatively, exemplary assay surfaces according to the disclosed subject matter can provide faster washing times, including when used with small sample volumes. By comparison, some conventional sample analysis systems can be unsuitable for use with reagent volumes less than 100 μL.

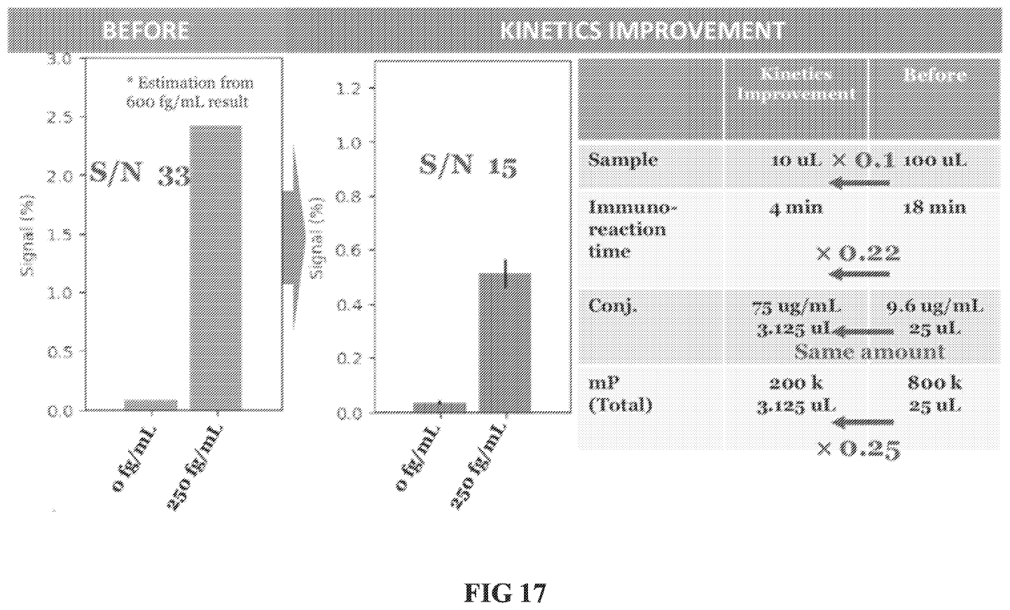

For purpose of illustration but not limitation, FIG. 17 shows exemplary results of an assay performed by a sample analysis system using an exemplary assay surface according to the disclosed subject matter with a sample volume of 10 μL compared to an assay performed by a conventional sample analysis system (e.g., Abbott ARCHITECT™) with a sample volume of 100 μL (according to the Instructions for Use) for purpose of illustration and confirmation of the disclosed subject matter.

Alternatively, the exemplary assay surface can have a sample volume less than 100 μL. For example and without limitation, the conventional HIV p24 assay was conducted with a 100-μL sample volume within a 18-minute immunoreaction time with 25 μL of 9.6 μg/mL conjugate and 25 μL of 800 k assay beads. For purpose of illustration of the disclosed subject matter, and without limitation, an HIV p24 assay was conducted with 10-μL sample volume using an assay surface according to the disclosed subject matter within 4-minute immunoreaction time with 3.125 μL of 75 μg/mL conjugate and 3.125 μL of 200 k assay beads.

Reducing the sample volume used with the conventional system from 100 μL to 10 μL (e.g., about 10 times) would be expected to result in a corresponding reduction of sensitivity of about 10 times (e.g., from a S/N of 33 to a S/N of less than 4). However, as shown in FIG. 17, the configuration using the 10-μL sample volume with the assay surface according to the disclosed subject matter achieved a S/N ratio of about 15, which is comparable to the S/N ratio of about 33 for the conventional system using a 100-μL sample and which can be suitable for optical detection including, but not limited to, analog or digital detection techniques described herein. The S/N ratio achieved using the 10-μL sample volume with the assay surface according to the disclosed subject matter can be due at least in part to kinetics improvements obtained during the immunoreaction occurring in the smaller sample volume. Providing a reduced sample volume prepared using less reagent volume to a concentration suitable for digital detections can allow for cost savings for each assay performed using systems for sample analysis according to the disclosed subject matter.

According to another aspect of the disclosed subject matter, an exemplary laboratory system, an assay processing unit (APU), or an assay processing system (APS) can be constructed. For purpose of illustration and not limitation, as embodied herein, exemplary sample analysis systems and methods can utilize exemplary assay surfaces described herein to achieve high-throughput, including but not limited to time per sample, samples over time, and samples over time per area (footprint) of the system.

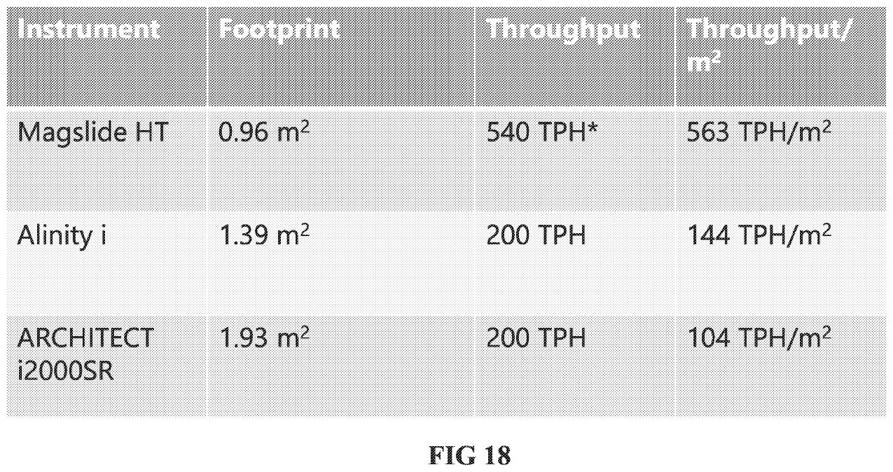

FIG. 18 illustrates additional details of an exemplary laboratory system including a plurality of APSs disclosed herein compared to conventional sample detection systems (e.g., Abbott Alinity i and Abbott ARCHITECT™ i2000SR) for purpose of illustration and confirmation of the disclosed subject matter. As shown in FIG. 18, for purpose of illustration not limitation, the exemplary laboratory system can achieve a throughput per area of about 560 tests per hour per square meter with a much smaller footprint of 0.96 square meter for the core sample preparation and detection components. For purpose of illustration not limitation, the exemplary laboratory system can include one or more exemplary APSs, and a controller configured to control a plurality of the one or more APSs to process a corresponding sample and to detect a presence of at least one corresponding analyte or determine a level or concentration of the at least one corresponding analyte substantially in parallel. The exemplary laboratory system can process multiple assay surfaces in a packed footprint. By comparison, the Abbott Alinity™ i system and the Abbott ARCHITECT™ i2000SR system have a throughput per area of about 140 tests per hour per square meter of footprint and 100 tests per hour per square meter of footprint, respectively.

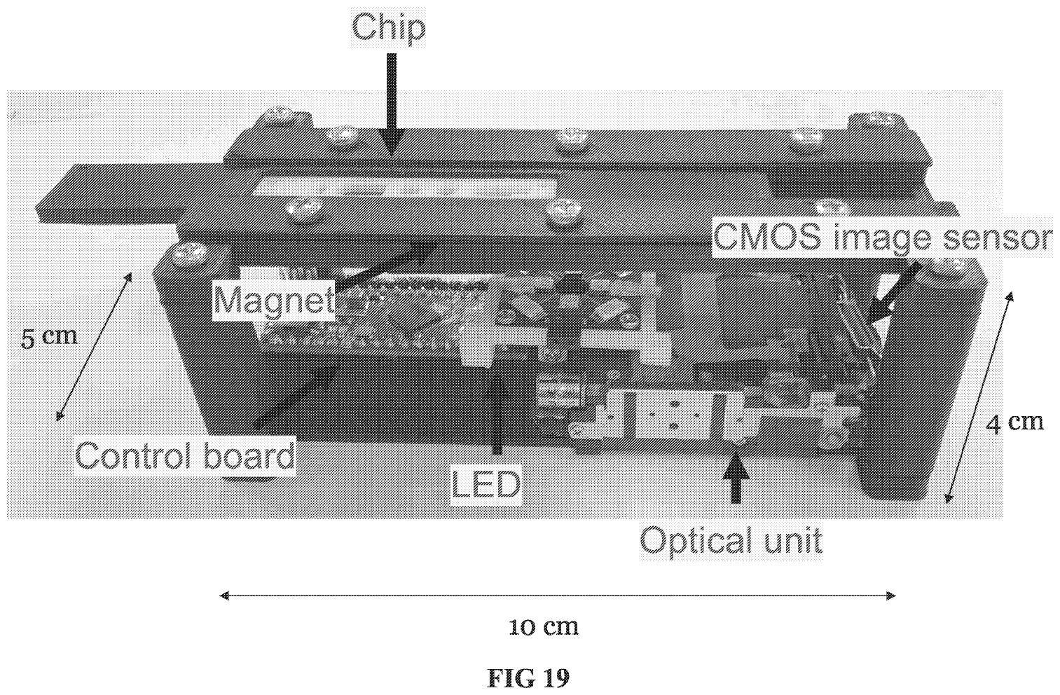

FIG. 19 illustrates additional details of an embodiment of an exemplary APS for use for an exemplary laboratory system and method for sample analysis having a throughput per area of about 560 tests per hour per square meter of footprint as shown in FIG. 18 and according to the disclosed subject matter, using assay surfaces described herein. For purpose of illustration not limitation, the exemplary laboratory system can include one or more exemplary APSs, and a controller configured to control a plurality of the one or more APSs to process a corresponding sample and to detect a presence of at least one corresponding analyte or determine a level or concentration of the at least one corresponding analyte substantially in parallel. The exemplary laboratory system can process multiple assay surfaces in a packed footprint. For purpose of illustration but not limitation, as shown for example in FIG. 19, as embodied herein, the exemplary APS can include one or more exemplary assay surfaces and an exemplary assay processing unit (APU). For purpose of illustration not limitation, the exemplary APU can include a control board comprising one or more processors configured to control the operations, LED lights, an optical unit, CMOS image sensor for detection, an assay surface receiving component, and a magnetic element to generate a magnetic field. For purpose of illustration and not limitation, exemplary processors recited herein can be configured to perform operations using hardware logic, firmware or software instructions. For purpose of illustration not limitation, the magnetic element can be an electromagnetic to generate a moving magnetic field, or a magnet operably connected with a sliding element. For purpose of illustration not limitation, the sliding element can be a motor. Additionally or alternatively, the magnetic element can be disposed in any suitable position relative to the assay surface received. As embodied herein, the exemplary APS in FIG. 19 is relatively compact yet performs tests with desired sensitivity in a short period of time, for example but not limitation, around 5.5 minutes. Packaging the APS of FIG. 19, as embodied herein having a footprint of about 0.005 square meters, together in multiples within one instrument, for example but not limitation, about 52, enables a high-throughput instrument as an exemplary laboratory system in a convenient footprint, as embodied herein of about 0.26 square meters.

For purpose of illustration and not limitation, as embodied herein, the exemplary assay processing system (APS) can include a receiving component as a process path to receive one or more assay surfaces to process the assay surfaces to shorten the total time-to-result for a sample to less than 6 minutes, and alternatively, a time-to-result can be between 3 to 5 minutes for one-step assays, or can be between 3 to 7 minutes for two-step assays. Alternatively, a time-to result can be between 2 to 5 minutes. Alternatively, a time-to result can be between 5 to 10 minutes. Alternatively, a time-to result can be less than 5 minutes. Alternatively, a time-to result can be less than 10 minutes. As embodied herein, an exemplary assay surface can enter a one-step assay receiving component of the APS. For purpose of illustration not limitation, there can be different receiving components (process paths) to accommodate different assay protocols. For example and without limitation, an exemplary assay surface (1200) or (1500) can be loaded from a storage unit of the exemplary APS. The sample can be added to the assay surface, for example by automatic or manual pipetting, or any other suitable technique, for about 10 seconds. For purpose of illustration not limitation, samples, microparticles, or reagents/conjugates can be stored on assay surfaces for use, or can be added manually or automatically from a reservoir using for example, pipetting, or other suitable techniques. A volume of liquid comprising the analyte can be prepared on the assay surface and various sample processing steps can be performed, mixing, washing, and/or incubation steps are performed, including, for example and without limitation, washing the sample-microparticle complex, adding conjugate to the sample, and adding a substrate to the sample. Oil can be added to the sample in one station and a first image can be captured under the control of the processer of the APU, which can be used to extend the dynamic range of detection at higher concentrations. The total sample processing time for the processes above can be about 3.5 minutes. An enzyme can be applied to the imaged sample after the first image, and the sample can be incubated for an enzyme reaction time to obtain a concentration suitable for digital detection. A plurality of images of the incubated sample can be obtained under the control of the processer, which can be used to determine a presence, absence or concentration of the analyte at lower concentrations. The total sample processing time through detection of the presence of the analyte in the sample is less than 6 minutes, and in some embodiments, a time-to-result can be between 3 to 5 minutes. For purpose of illustration not limitation, the table below summarizes one example of a one-step assay process that results in a test time of about 5.5 minutes. In the configuration of FIG. 19, packaged in a multiple of 52 within a single instrument for a laboratory system for parallel processing, together with associated sample, reagent and disposable handling systems, the total throughput per hour for the one-step assay process, as embodied herein, is about 572 tests per hour with the single instrument having about a 1 square meter 10 footprint. Additional units can be packaged within the same single instrument footprint to achieve more tests per hour, including 400, 500 or 600 tests per hour, or can be configured to achieve a throughput within a range of 375 to 600 tests per hour.

| | Duration | Cumulative | Cumulative | |
|---|---|---|---|---|
| | in | Time in | Time in | |
| Steps | Seconds | Seconds | Minutes | Description |
| 1 | 20 | 20 | .33 | Load vessel and |
| | | | | dispense sample |
| 2 | 200 | 220 | 3.67 | Incubate and Wash - |
| | | | | 3.5 minutes total |
| | | | | duration from sample |
| | | | | apply to the oil |
| | | | | sealing. |
| | | | | Various solution |
| | | | | mixing and B/F |
| | | | | separation steps occur, |
| | | | | including washes, conj. |
| | | | | addition, loading of |
| | | | | digital detector |
| 3 | 10 | 230 | 3.83 | Oil addition + 1^{st} Image - |
| | | | | 1st image for high end |
| | | | | (dynamic) range |
| 4 | 80 | 310 | 5.17 | Enzyme Incubation |
| 5 | 10 | 320 | 5.33 | 2^{nd} Image - 2nd image |
| | | | | (for low end range data |
| | | | | point) |
| 6 | 10 | 330 | 5.5 | Vessel disposal to waste |

For purpose of illustration but not limitation, alternatively or additionally, two-step assays can be performed on a process path. A time-to-result can be between 3 to 7 minutes. Alternatively, a time-to-result can be less than 5 minutes. Alternatively, a time-to-result can be less than 10 minutes. The table below summarizes one example of a two-step assay process that results in a test time of about 7 minutes. For purpose of illustration not limitation, samples, microparticles, or reagents/conjugates can be stored on assay surfaces for use, or can be added manually or automatically from a reservoir using for example, pipetting, or other suitable techniques. In the configuration of FIG. 19, exemplary APSs packaged in a multiple of 67 within a single instrument in an exemplary laboratory system, together with associated sample, reagent and disposable handling systems, the total throughput per hour for the two-step assay process, as embodied herein, is about 570 tests per hour with the single instrument having about a 1 square meter footprint. Additional units can be packaged within the same footprint in a laboratory system to achieve more tests per hour, including 400, 500 or 600 tests per hour, or can be configured to achieve a throughput within a range of 375 to 600 tests per hour.

| | Duration | Cumulative | Cumulative | |
|---|---|---|---|---|
| | in | Time in | Time in | |
| Steps | Seconds | Seconds | Minutes | Description |
| 1 | 20 | 20 | .33 | Load vessel and |
| | | | | dispense sample |
| 2 | 200 | 220 | 3.67 | Incubate and Wash - |
| | | | | 3.5 minutes total |
| | | | | duration from sample |
| | | | | apply to the oil |
| | | | | sealing. |
| | | | | Various solution |
| | | | | mixing and B/F |
| | | | | separation steps occur, |
| | | | | including washes, conj. |
| | | | | addition |
| 2a | 90 | 310 | 5.17 | Incubate and Wash - |
| | | | | 2^{nd} immunoreaction, |
| | | | | including wash, loading |
| | | | | of digital detector |
| 3 | 10 | 320 | 5.33 | Oil addition + 1^{st} Image - |
| | | | | 1st image for high end |
| | | | | (dynamic) range |
| 4 | 80 | 400 | 6.67 | Enzyme Incubation |
| 5 | 10 | 410 | 6.83 | 2^{nd} Image - 2nd image |
| | | | | (for low end range data |
| | | | | point) |
| 6 | 10 | 420 | 7 | Vessel disposal to waste |

For purpose of illustration not limitation, exemplary laboratory systems can be configured to perform one or more of an HIV p24 assay, an HBsAg assay, a Troponin I assay, a TSH assay, a Myoglobobin assay, a PSA assay, a BNP assay, a PIVKA-II assay, an HIV Ab assay, an estradiol assay, a COVID-Ag assay, and other assays.

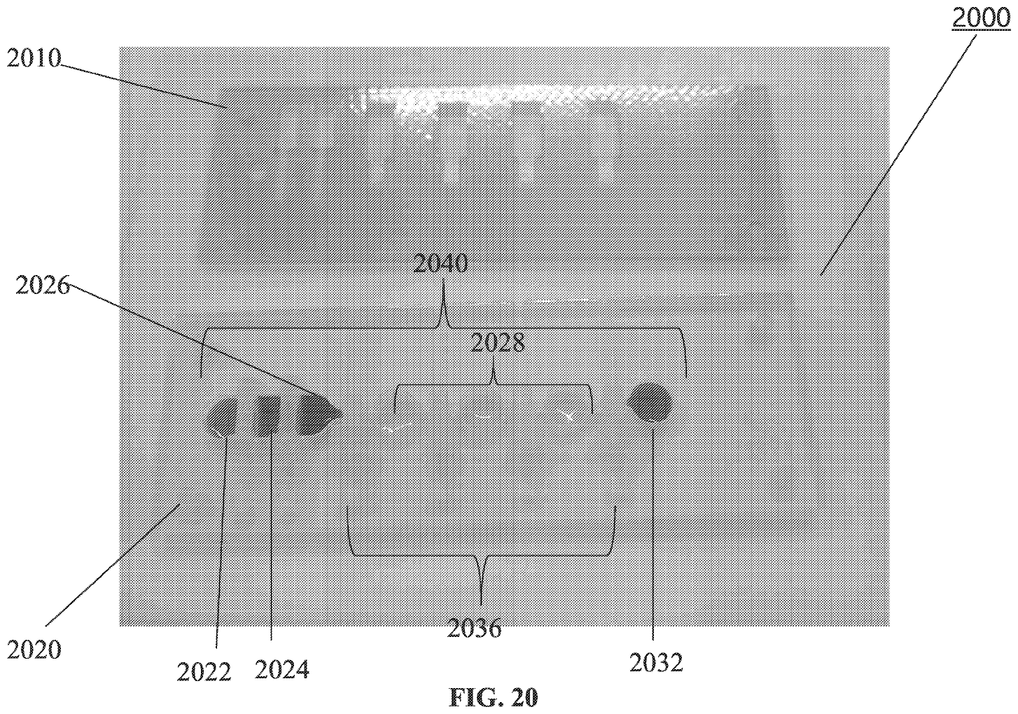

FIG. 20 illustrates an exemplary assay surface (2000) of the disclosed systems and methods for preparing and detecting an analyte of interest in a sample. As illustrated, an exemplary assay surface (2000) can include an upper portion (2010) and a lower portion (2020). The upper portion (2010) can cover and seal the lower portion (2020) when preparing and detecting an analyte of interest. For purpose of illustration not limitation, an exemplary assay surface can include a plurality of regions and a plurality of channels in the lower portion (2020), each of which can be arranged in a series to define a sample preparation and detection area (2040). As embodied herein, the exemplary assay surface (2000) can include a sample preparation and detection area (2040) having a microparticle storage region (2022), a sample storage region (2024), a sample/conjugate mixing region (2026), one or more wash regions (2028), and a detection region (2032). As embodied herein, the exemplary assay surface (2000) has three wash regions (2028). As embodied herein, the surface of the lower portion (2020) can be made of a hydrophobic material, for example, COP.

For purpose of illustration not limitation, as embodied herein, the microparticle storage region (2022) can include a plurality of microparticles. Alternatively, the microparticles can be loaded into the region manually or automatically using, for example, a pipettor, from a microparticle reservoir. As described herein, the microparticles can be magnetic or paramagnetic to facilitate the use of magnet forces to perform the sample analysis and detection. Additionally or alternatively, the magnetic or paramagnetic beads or particles can specifically bind to an analyte of interest or a reagent/conjugate. The microparticles can travel through regions of the exemplary assay surface under a magnetic force. For purpose of illustration, the magnetic force can be a magnetic field generated by an exemplary assay processing unit (APU) disclosed herein.

Additionally or alternatively, the sample storage region (2024) can include analytes of interest for preparation and detection in a suitable solution. As embodied herein, an analyte of interest can be, for example, an HIV Ab p24 assay, an HIV1-Ab assay, an HBsAg assay, or a COVID-Ag assay. Alternatively, the analyte of interest can include other analytes.

For purpose of illustration not limitation, the sample/conjugate mixing region (2026) can be configured for mixing the analytes of interest with the microparticles and/or reagents/conjugates. As embodied herein, reagents or conjugates can be stored in the mixing region (2026). Alternatively, reagents or conjugates can be loaded to the region manually or automatically using, for example, a pipettor, from a larger reservoir. For purpose of illustration and not limitation, as embodied herein, an analyte of interest of HIV Ab p24 assay can be mixed with paramagnetic beads (800 k beads) and enzyme nCIAP-anti p24 conjugates.

Furthermore, one or more wash regions (2028), if provided, can be sized to contain one or more wash buffers to remove any unbound analytes of interest. As embodied herein, wash regions can be used to remove any molecules not bound with any microparticles. The exemplary assay surface (2000) can include any number of wash regions, which as embodied herein, can include three wash regions. In exemplary assays described herein, the wash period for each wash region can be approximately 90 seconds.

For purpose of illustration not limitation, the detection region (2032) can be configured for detecting an analyte of interest. The detection region (2032) can be configured for analyte detection using any analyte detection technique described herein. For example and without limitation, exemplary analyte detection techniques can include one or more of optical detection, analog signal detection, digital signal detection, illumination detection, fluorescence detection, or any combination of these techniques. Additionally or alternatively, the detection region (2032) can be configured to perform single-molecule counting. Furthermore, for purpose of illustration not limitation, the detection region can include a plurality of elements, each dimensioned to hold at least one single bead or particles. As embodied herein, the array of elements can include an array of nanowells configured for detection, by separating microparticles bound with analytes of interest into the plurality of nanowells. For purpose of illustrations, as embodied herein, the microparticles or beads can be loaded into the plurality of nanowells using magnetic force. Using magnetic force to load microparticles into the plurality of nanowells can improve loading efficiency and accuracy. For purpose of illustration not limitation, as embodied herein, most of the array of nanowells can be loaded with at least one microparticle, which may also improve the efficiency of single-molecule detection.

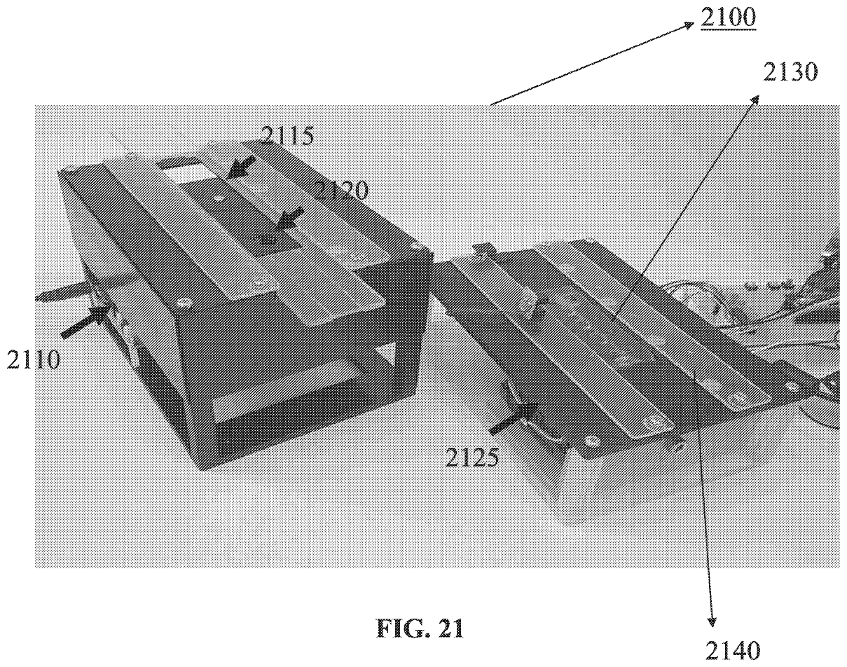

FIG. 21 illustrates a front perspective view of an exemplary assay processing unit (APU) (2100) for preparing and detecting an analyte of interest in a sample using an exemplary assay surface in an assay processing system (APS) according to the disclosed subject matter. For purpose of illustration not limitation, as embodied herein, the exemplary APU (2100) can include a processor (2110), a magnetic element (2115), a detection region (2120), an assay surface receiving component (2150), and a detection component (2125). An exemplary APS can include one or more exemplary assay surfaces an exemplary APU.

Referring still to FIG. 21, the processor (2110) can include a control board configured to control the operations to be performed on an assay surface, detection component (2125) and movements of other components of the exemplary APU (2100). For purpose of illustration not limitation, the processor (2210) can include an Arduino Micro computer system. As embodied herein, the detection component (2125) can include a camera and a light source, such as an LED, arranged to conduct optical detection of an analyte of interest. Alternatively, the detection component can include other suitable instruments for other types of detections. As embodied herein, the assay surface (2130) received in the assay surface receiving component (2150) can be an exemplary assay surface (2000) as disclosed above. Alternatively, the assay surface (2130) received can be other assay surfaces.

For purpose of illustration not limitation, the magnetic element (2115) of the exemplary APU can include an electromagnet generating a moving magnetic field. Alternatively, as embodied herein, the magnetic element (2115) can include a magnet operably connected with a sliding mechanism (2140). The sliding mechanism (2140) can be controlled by the processor (2110) and can move the magnet in a horizontal direction, for example, with a motor. Additionally or alternatively, the magnetic element (2115) can be disposed at any suitable location relative to the assay surface (2130) received. For example, the magnetic element (2115) can be below or above the assay surface (2130), or near a side of the assay surface (2130). For purpose of illustration only, FIG. 21 depicts the magnetic element (2115) below the assay surface (2130).

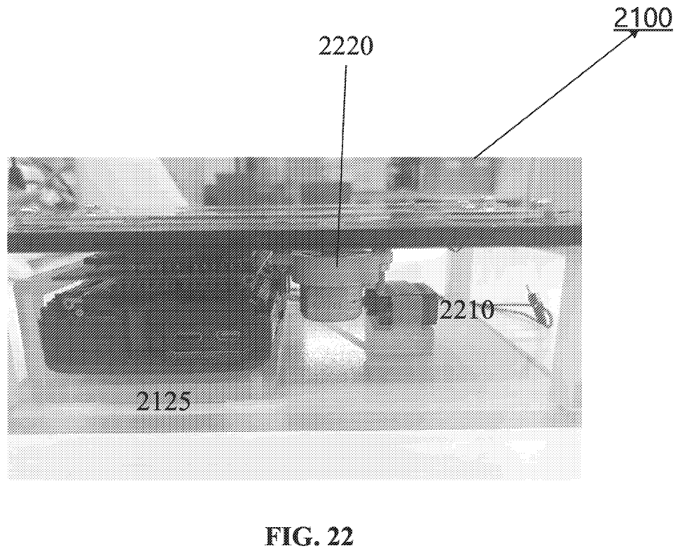

FIG. 22 illustrates a side view of the exemplary APU (2100) of FIG. 21. As embodied herein, the APU (2100) can include the detection component (2125), a drive element (2210), and a stepping motor (2220) for the sliding mechanism (2140). For purpose of illustration not limitation, the magnetic element (2115) can be an electromagnet generating a moving magnetic field in a horizontal or vertical direction defined by a top surface of the assay surface received. Alternatively, as embodied herein, the magnetic element (2115) can be a magnet. A drive element (2210) can operably connected to the magnet to cause the magnet to move in a vertical direction defined by a top surface of the assay surface received. For purpose of illustration, the drive element (2115) can be a motor or a string. Additionally or alternatively, the APU can also include a mixing dynamics component, which can include electromagnets, ultrasonic mixing elements, ballistic mixing elements with a pipettor, or other suitable elements to improve the mixing frequency. The mixing dynamics component can cause at least one volume of liquid disposed on the assay surface received in the APU and APS to mix under a predetermined frequency. For purpose of illustration not limitation, as embodied herein, the mixing dynamics element can be a vibration motor.

For purpose of illustration not limitation, the detection component (2125) can be configured for detection an analyte of interest using optical detection, and can include, for example, a camera and a light source, such as an LED. For purpose of illustration not limitation, as embodied herein, when the magnetic element (2115) is a magnet, the drive element (2210) can be connected to the magnet with a nut-bolt connection and can move the magnet toward and away from the assay surface in a vertical direction perpendicular to a plane defining a top surface of the assay surface received. Alternatively, the magnetic element (2115) can be an electromagnet generating a moving magnetic field in a vertical direction. As embodied herein, the movement direction of the magnet is perpendicular to a plane defined by a top surface of the assay surface (2130) received.

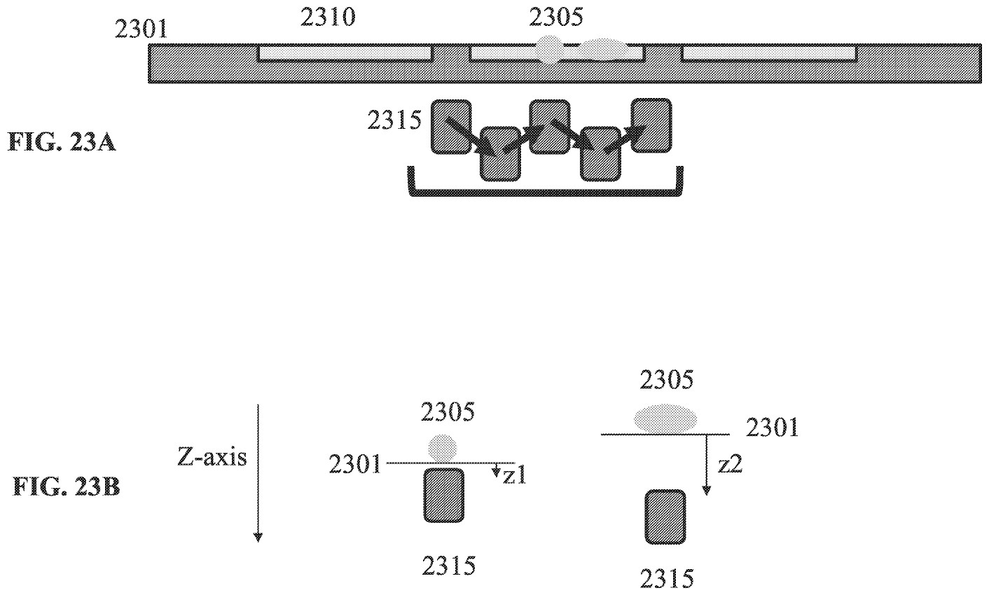

FIGS. 23A and 23B illustrate movements of a plurality of microparticles in a droplet in a wash region of an exemplary assay surface under magnetic force of a magnetic element, and for purpose of illustration only, other regions and features of the assay surface described herein are omitted in this schematic drawing. As embodied herein, an exemplary assay surface (2301) can include three wash regions (2310). A magnetic element (2315) can generate a moving magnetic field in a vertical direction from a plane defined by a top surface of the assay surface (2301). Additionally or alternatively, the magnetic element (2315) can be disposed at any suitable locations, for example, above or below the assay surface (2301), or by a side of the assay surface (2301). As embodied herein, for purpose of illustration, the magnetic element (2315) can be a magnet disposed below the assay surface (2301). The magnet can be connected to a drive element (not depicted in the figure), for example, a motor. The drive element can cause the magnet to move toward and away from the assay surface in a vertical direction. Alternatively, the magnetic element (2315) can be one or more electromagnets generating a moving magnetic field in a vertical direction. Furthermore, as embodied herein, a droplet with microparticles (2305) is in one of the wash regions. The droplet can include microparticles to be moved under magnetic force of the magnetic element (2315).

As illustrated in FIG. 23B, as embodied herein, when the magnet is moved closer to the assay surface (2301), the droplet (2305) with microparticles can be drawn toward and relatively accumulate closer to the magnet. When the magnet is moved away from the assay surface (2301), the droplet (2305) with microparticles can relatively spread away from each other under less force from the magnet. For purpose of illustration not limitation, the magnet can be moved away from a lower surface of the assay surface (2301) for approximately 5 mm. Alternatively, the moving magnetic field can be generated by an electromagnet without changing the position of the electromagnet. For purpose of illustration, as embodied herein, when the magnet is the closest to the assay surface (2301), a distance between the assay surface (2301) and the magnet Z1 can be about 0 mm. For purpose of illustration, as embodied herein, when the magnet is the furthest from the assay surface (2301), a distance between the assay surface (2301) and the magnet Z2 can be about 5 mm. Furthermore, as embodied herein, for each wash region, the magnet can be moved upwards and downwards for approximately 4 times to improve wash efficiency. Alternatively, the moving magnetic field by a magnetic element (2315) can be generated by an electromagnet controlled by one or more processors of the APU.

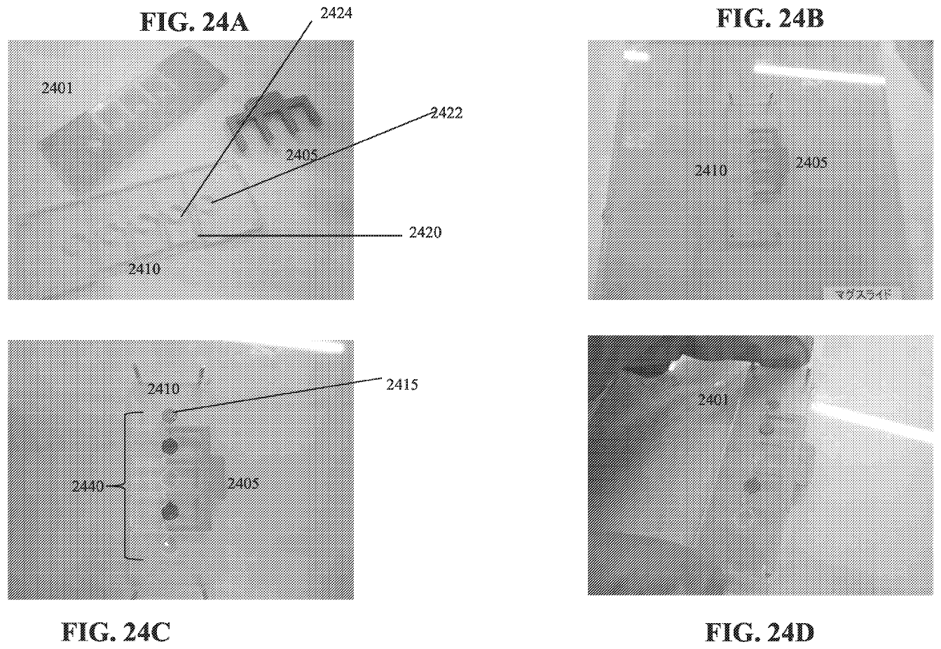

FIGS. 24A-24D illustrate an alternative exemplary assay surface with a plurality of stopping elements. As embodied herein, an exemplary assay surface can include an upper portion (2401), a lower portion (2410), and a plurality of stopping elements (2405). As illustrated in FIG. 24A and embodied herein, for purpose of illustration not limitation, the lower portion (2410) can include a plurality of regions and a plurality of channels defining a sample preparation and detection area (2440) as disclosed in accordance with the subject matter. For example, a channel (2420) is in between a first region (2422) and a second region (2424). For purpose of illustration not limitation, the first region can be configured for storing microparticles, and the second region can be configured for storing an analyte of interest in suitable solutions. Alternatively, microparticles or analytes can be loaded manually or automatically from a reservoir. As embodied herein, a surface of the lower portion (2410) can include a hydrophobic material, for example, COP, as described herein.

As illustrated in FIG. 24B, for purpose of illustration not limitation, the plurality of stopping elements (2405) can be inserted into the plurality of channels in the lower portion (2410). As illustrated in FIG. 24C, as embodied herein, the plurality of regions of the lower portion (2410) can include solutions and droplets for preparation and detection an analyte of interest. Additionally or alternatively, a region (2415) can include a plurality of microparticles to bind with an analyte of interest. As illustrated in FIG. 24D, the upper portion (2401) can cover the lower portion (2410) and the plurality of stopping elements (2405) to define the reaction area, and can be joined to seal and protect the reaction area from unwanted ingress or egress of substances into and out of the reaction area.

For purpose of illustration not limitation, the plurality of stopping elements (2405) can be made of a hydrophobic material, for example, rubber. Different compositions or solutions can be stored in the plurality of regions in an assay surface, and when the plurality of stopping elements (2405) is disposed in the plurality of channels, the plurality of stopping elements can prevent or inhibit unwanted movement of the contents of the regions into different regions, for example and without limitation during shipment, storage, and handling of the assay surface.

Referring now to FIGS. 20-22, an exemplary sample preparation and detection system (an assay processing system (APS)) and method are disclosed with reference to and using an exemplary assay surface (2000) and an exemplary APU (2100). Alternatively, an APS can include alternative assay surfaces and alternative APUs. For purpose of illustration not limitation, as embodied herein, an analyte of interest can be an HIV Ag p24 assay. First, suitable solutions are loaded into a lower portion (2020) of the assay surface (2000). As embodied herein, for purpose of illustration not limitation, a suitable solution can be a serum specimen.

As embodied herein, a microparticle storage region (2022) can include paramagnetic beads that can bind with HIV Ag assay, for example, MS 300 beads. Alternatively, the microparticles can be loaded manually or automatically from a reservoir. A sample storage region (2024) can include an assay of HIV Ag p24 in a suitable solution. A sample/conjugate mixing region (2026) can include suitable conjugates and reagents for immunoreactions and/or enzyme reactions, for example, enzyme nCIAP-anti p24 conjugate (1 AP/conjugate). Alternatively, the reagents/conjugates can be loaded manually or automatically from a larger reservoir. The total solution volume for the microparticle storage region (2022), the sample storage region (2024), and the sample/conjugate mixing region (2026) can be about 15 μL. The total volume capacity for the microparticle storage region (2022), the sample storage region (2024), and the sample/conjugate mixing region (2026) can be about 25 μL or less. A plurality of wash regions (2028) can each include about 10 μL wash buffer. A detection region (2032) can include a plurality of elements, each dimensioned to hold at least a single one of microparticles. As embodied herein, the detection region (2032) can include an array of nanowells configured for analyte detection. The detection region (2032) can include 50 μL AP's substrate buffer. After the assay surface (2000) is loaded, a plurality of stopping elements can be inserted into a plurality of channels (2036) and an upper portion (2010) can cover the lower portion with the plurality of stopping elements.

The assay surface (2000) can be disposed on an assay surface receiving component in the exemplary APU (2100). The magnetic element (2115) can generate a moving magnetic field. For purpose of illustration not limitation, as embodied herein, a magnetic element (2115) can include a magnet, and a sliding mechanism (2140) can cause the magnet to move in a horizontal direction.

Two of the plurality of stopping elements can be removed from a channel between the microparticle storage region (2022) and the sample storage region (2024), and a channel between the sample storage region (2024) and the sample/conjugate mixing region (2026). A mixing dynamics element, for example, a vibration motor, if included in the APU can cause solutions and droplets in the microparticle storage region (2022), the sample storage region (2024), and the sample/conjugate mixing region (2026) to vibrate at a predetermined frequency, which can facilitate the paramagnetic beads to bind with the analyte of interest, HIV Ag p24. As embodied herein, the vibration motor can vibrate the solutions in the regions for approximately 110 seconds to sufficiently perform immunoreactions. Alternatively, the mixing dynamics element can include an electromagnet to facilitate mixing under a magnetic field. For purpose of illustration not limitation, for the analyte of interest, HIV Ag p24, after the mixing and enzyme reaction, positive and negative signals received in a detection region of the exemplary assay surface are comparable to those received based on a manual assay.

As embodied herein, a stepping motor (2220) can move the magnet from the sample/conjugate mixing region (2026) to a first wash region (2028). Alternatively, the magnetic element (2115) can be one or more electromagnets generating a moving magnetic field along a length of the assay surface. One of the plurality of stopping elements (not depicted in the figure) can be removed from a channel in between the sample/conjugate mixing region (2026) and a first region of a plurality of wash regions (2028). As embodied herein, a drive element (2210) connected to the magnet can cause the magnet to move closer to and away from the first wash region. As embodied herein, the magnet can move upwards and downwards 4 times. Alternatively, this can be achieved by one or more electromagnets generating a moving magnetic field in a vertical direction. Similar techniques can be performed again as described herein, for example for a second and third and any additional wash regions to prepare the sample for detection. After washing, paramagnetic beads bound with HIV Ag p24 can be moved into the detection region (2032). As embodied herein, the detection region (2032) can include an array of nanowells. For purpose of illustration, loading the microparticles or beads into the nanowells can be under magnetic force. The magnetic force can be generated by the magnetic element (2115) of the exemplary APU. The magnetic element can be a magnet or an electromagnet. For purpose of illustration, loading beads or microparticles under magnetic force can improve the efficiency and accuracy. Additionally, multiple passes or movements of the microparticles over the detection region (2032) can increase the loading percentage in the plurality of nanowells. Additionally or alternatively, an inert liquid, for example, oil can be dispensed to seal the plurality of nanowells for detection. As embodied herein, the plurality of nanowells can be sealed by approximately 150 μL oil dispensed from an oil storage (not depicted in the figure), for example, a syringe oil pump.

Referring still to FIGS. 20-22, as embodied herein, the detection region (2032) of the assay surface (2000) can be imaged in a detection region (2120) of the APU by a detection component (2125) of the APU. For purpose of illustration not limitation, the detection component (2125) can include a camera configured to record or measure optical signals from the plurality of nanowells with the analyte of interest and microparticles inside. For purpose illustration not limitation, one or more processors of the exemplary APU can cause the detection component (2125) to obtain a series of images of the detection region (2032) of the exemplary assay surface. As embodied herein, the detection component (2125) can count individual signals from each of the plurality of nanowells or surface of a bead in each of the plurality of nanowells to perform single-molecule counting every 30 seconds. Alternatively or additionally, the detection component (2125) can measure an intensity of optical signals representative of the presence or concentration of the analyte in the nanowells.

For purpose of illustration not limitation, as embodied herein, for an HIV Ag p24 assay (600 fg/ml), after 2 minutes of immunoreaction and 1.5 minutes of enzyme reaction under 37° C., an exemplary system as disclosed above can achieve an equivalent detection sensitivity compared to a conventional sample preparation and detection device, for example, Abbott ARCHITECH™ systems. For purpose of illustration not limitation, the total assay preparation time for the HIV Ag p24 assay can be approximately 5.5 minutes. For an HIV1-Ab assay (0.02 dil.), after 2 minutes of immunoreaction and 3 minutes of enzyme reaction under 37° C., an exemplary system as disclosed above can achieve an equivalent detection sensitivity compared to a conventional sample preparation and detection device, for example, Abbott ARCHITECH™ systems. For purpose of illustration not limitation, the total assay preparation time for the HIV1-Ab assay can be approximately 7 minutes. For an HBsAg assay (1 fM), after 2 minutes of immunoreaction and 2 minutes of enzyme reaction under 37° C., an exemplary system as disclosed above can achieve an equivalent detection sensitivity compared to a conventional sample preparation and detection device, for example, Abbott ARCHITECH™ systems. For a COVID-Ag assay (10,000 cp/ml), after 2 minutes of immunoreaction and 1.5 minutes of enzyme reaction under 37° C., an exemplary system as disclosed above can achieve an equivalent detection sensitivity compared to a conventional sample preparation and detection device, for example, Abbott ARCHITECH™ systems. For purpose of illustration not limitation, the total assay preparation time for the COVID-Ag assay can be approximately 5.5 minutes. As embodied herein, for purpose of illustration not limitation, a sample volume for the exemplary system can be 10 μL, and a reagent assay volume for the exemplary system can be 15 μL. Alternatively, a sample volume for the exemplary system can be between about 10 μL and about 50 μL. Alternatively, a sample volume for the exemplary system can be less than 50 μL. Alternatively, a sample volume for the exemplary system can be less than 75 μL. Alternatively, a sample volume for the exemplary system can be less than 100 μL.

Alternatively, as embodied herein, for an HIV Ag p24 assay (600 fg/ml), an exemplary system can achieve equivalent detection sensitivity compared to a conventional sample preparation and detection device, for example, Abbott ARCHITECH™ systems, with a total of 5.5 minutes of assay preparation time, including 2 minutes of immunoreaction and 1.5 minutes of enzyme reaction. As embodied herein, for a COVID-Ag assay (10,000 cp/ml), an exemplary system can achieve equivalent detection sensitivity compared to a conventional sample preparation and detection device, for example, Abbott ARCHITECH™ systems, with a total of 5.5 minutes of assay preparation time, including 2 minutes of immunoreaction and 1.5 minutes of enzyme reaction.

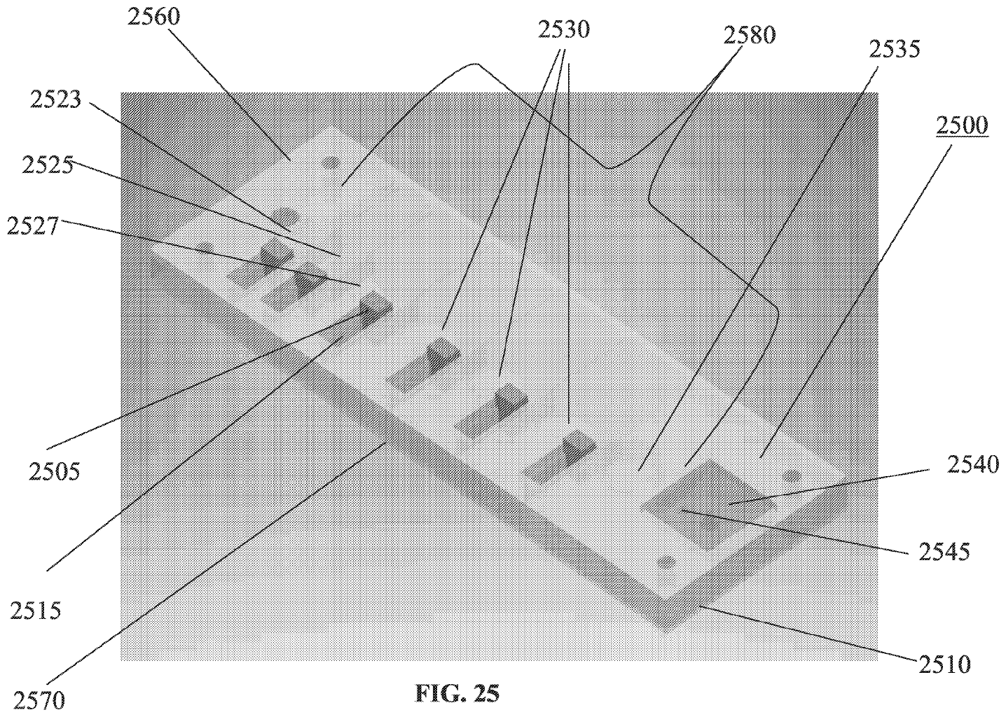

FIG. 25 illustrates an exemplary assay surface (2500) according to the disclosed subject matter. As embodied herein, an upper portion (2560) of the assay surface (2500) covers a plurality of stopping elements (2505) and a lower portion (2570) of the assay surface (2500). As embodied herein, the lower portion (2570) can include a plurality of regions and a plurality of channels defining a sample preparation and detection area (2580). For example, the plurality of stopping elements (2505) is disposed in one channel (2515).

As embodied herein, a microparticle storage region (2523) of the lower portion (2570) can be configured to store a plurality of microparticles. Alternatively, the microparticles can be loaded to the region (2523). A sample storage region (2525) of the lower portion (2570) can be configured to store an analyte of interest in a suitable solution. A sample/conjugate mixing region (2527) of the lower portion (2570) can be configured for mixing a sample with the microparticles and reagents and/or conjugates. Alternatively, reagents/conjugates can be added to the region (2527). As embodied herein, the lower portion (2570) can include one or more wash regions (2530). For purpose of illustration not limitation, the lower portion (2570) includes three wash regions. As embodied herein, the lower portion can include a detection region (2535) configured for detecting the analyte of interest. Additionally or alternatively, the detection region can include an optical detection component, a plurality of nanowells configured for analyte digital detection, or any other suitable detection component. For purpose illustration not limitation, when conducting sample analysis, the microparticles can be moved under magnetic force through the regions and into the detection region (2535).

As embodied herein, the assay surface can further include an inert liquid storage region (2540). The inert liquid storage region can be configured to disperse an inert liquid, for example, an oil, to seal at least one of the plurality of regions. Additionally or alternatively, the inert liquid storage region (2540) can include a liquid inlet (2545) to dispense the liquid.

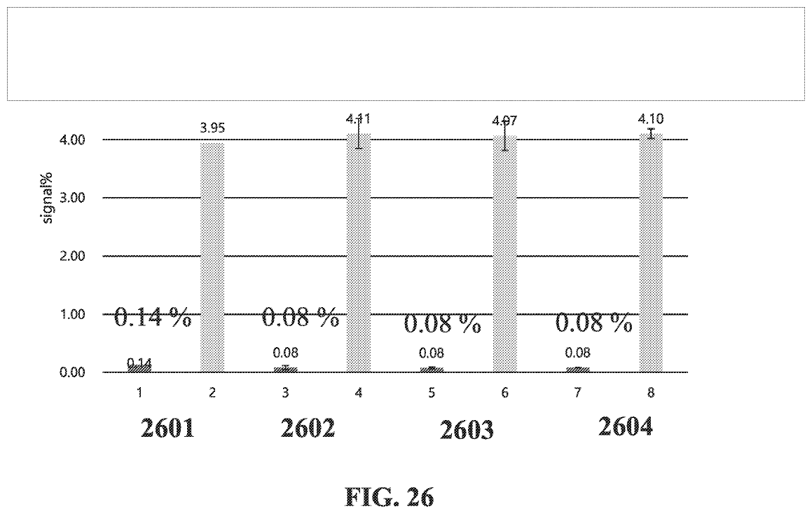

FIG. 26 is a chart illustrating washing efficiency using washing techniques with a moving magnetic field as described herein for purpose of confirmation of the disclosed subject matter and comparison to a King-Fisher wash technique. For purpose of illustration not limitation, as embodied herein, an analyte of interest of HIV Ab p24 assay is analyzed. A sample droplet can be mixed with paramagnetic beads, for example, MS 300 beads. Each evaluation of the washing efficiency can include two bars in the chart. For example, bar 1 and bar 2 represent the negative signals and positive signals received during detection. For purpose of illustration not limitation, the bars with odd numbers (1, 3, 5, and 7) represent negative signals received during each evaluation. When the negative signal received is lower, the washing efficiency is higher. For purpose of illustration, evaluation 2601 represents the signals received without movements of the magnetic field, and evaluation 2602 represents the signals received after movements of the magnetic field in a vertical direction. For purpose of illustration, percentage of signal is a unit of measurement when digital detection is performed, which can be calculated by the numbers of positive nanowells divided by the numbers of microparticles in the detection area. Evaluation 2601 received 0.14% signal, and evaluation 2602 received 0.08% signal. For purpose of illustration, evaluations 2603 and 2604 are for washing using King-Fisher method one time and three times, respectively. Each of them received 0.08% signal. For purpose of illustration not limitation, washing a mixed sample droplet using movements of the magnetic field in a vertical direction improves the washing efficiency to an equivalent level to King-Fisher washing method.

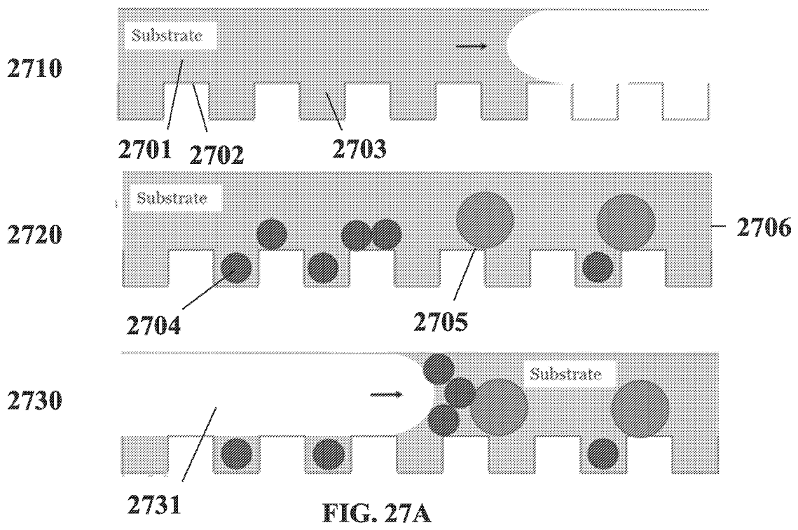

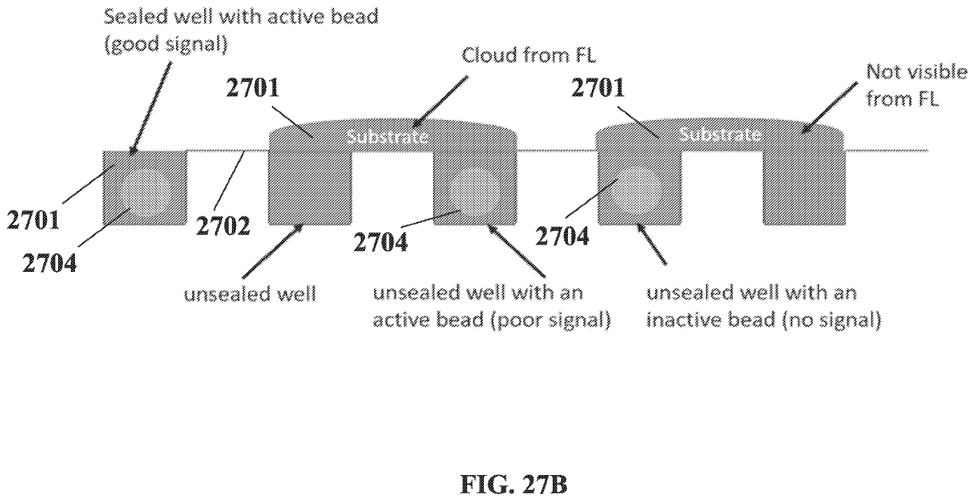

FIGS. 27A-27B are diagrams illustrating exemplary bead loading techniques using a moving magnetic field as described herein. As embodied herein, the exemplary techniques can involve seeding and sealing a single bead or microparticle that is conjugated with an analyte and enzyme sandwich, as described herein, into a single well filled with a chemical solution (also referred to herein as a substrate).

As shown for example in FIG. 27A, an exemplary bead loading technique 2700 can include substrate filling 2710, microparticle seeding 2720, and inert liquid sealing 2730. At 2710, a substrate solution 2701 is prepared, which can include a phosphate-based material and a wash buffer. The substrate 2701 can be configured to activate a fluorescent signal from microparticles 2704 containing the analyte of interest for detection as discussed further herein. With reference to FIG. 27A, for purpose of illustration and not limitation, substrate solution 2701 is introduced into the region proximate to the microwell array 2702 prior seeding of the microparticles 2704. As embodied herein, the area above the microwell array 2702 is flooded with substrate solution 2701, which fills one or more of the microwells of the microwell array 2702. The surface of microwell array 2702 can be hydrophilic to substrate solution 2701 such that each microwell 2703 is filled with substrate 2701 when introduced to the microwell array 2702. The wetting of the surface of microwell array 2702 with the substrate liquid can facilitate movement of microparticles 2704 across the microwell array surface 2702 as discussed further herein.

Referring still to FIG. 27A, at 2720, microparticles 2704 are seeded into microwell arrays. A magnet (not shown) brings conjugated microparticles 2704, having a size to fit within the microwells, along with assisting microparticles 2705 (if used), into a microwell array chamber 2706. Assisting microparticles 2705 can be provided to assist with microparticle transportation. For purpose of illustration only not limitation, as embodied herein, assisting microparticles 2705 can have a size of 10 μm, which is larger than the 2.8 μm functional microparticles 2704 and 4.5 μm microwells 2703 in this example, thus preventing assisting microparticles 2705 from becoming seeded within the microwells 2703. The magnetic field can be configured to introduce most or all of the microparticles 2704 into microwells 2703 and/or to remove remaining microparticles 2704 and assisting microparticles 2705 (if used) from the array surface 2702. After microparticles 2704 are seeded into the microwells 2703, some excess microparticles 2704 and/or assisting microparticles 2705 (if used) can remain on the top surface 2702 of the array.

With continued reference to FIG. 27A, at 2730, inert liquid sealing occurs in which an inert liquid, such as oil, is introduced into the microwell chamber to form an array of sealed microwells containing an amount of substrate fluid and a single microbead 2704. For example and without limitation, as embodied herein, the inert liquid or oil 2731 can include Fluorinert™ FC-40 by 3M. As embodied herein, oil 2731 can be pulled into the microwell chamber with negative pressure from a negative pressure source disposed at the opposite side of the chamber from which the oil is introduced. Oil 2731 can further remove the excess microparticles 2704 and assisting microparticles 2705 (if used) from the top of the microwell array and seal individual microwells 2703 prior to performing detection, including any incubation (if performed) and imaging of the microparticles 2703 as described herein. Parameters that can be affect oil sealing 2730 include the magnetic field applied during seeding 2720, the surface property of the microwells array 2702, liquid property of the substrate 2701 and oil 2731, amount of the functional microparticles 2704 and assisting microparticles 2705 (if used), seeding efficiency, and oil sealing flow rate.

Referring now to FIG. 27B, an example of a microwell array having sealed wells and unsealed wells is shown to further illustrate the benefit of exemplary bead loading techniques including seeding and sealing of the wells as described herein. At the left, a sealed well filled with substrate 2701 is shown containing a single active microparticle 2704. The substrate 2701 can be configured to activate a fluorescent signal from the active microparticle 2704 containing the analyte of interest for detection, and the sealed well can isolate the microparticle 2704 from others to allow the single microparticle 2704 containing a single molecule of the analyte of interest to be counted.

Referring still to FIG. 27B, to the right of the sealed well, a first pair of unsealed wells is shown with substrate 2701 pooling above the microarray surface 2702 and between the pair of unsealed wells, one of which contains a single active microparticle 2704. As such, when the fluorescent signal from the active microparticle 2704 is activated by the substrate 2701, the fluorescent signal can leak into the pool of substrate 2701 above the array surface 2702 and toward the adjacent well causing a poor fluorescent signal to be detected. The poor fluorescent signal can prevent the single molecule of the analyte of interest from being counted, for example due to noise or interference introduced by the leaking of the fluorescent signal out of the microwell containing the particle and onto the surface of the microarray 2702 and/or adjacent wells. For example, and not limitation, pooling of the substrate can cause the resulting image of the array to be hazy or cloudy, which can prevent or inhibit determination of whether individual microparticles 2704 are active.

With continued reference to FIG. 27B, to the right of the first pair of unsealed wells, a second pair of unsealed wells is shown with substrate 2701 pooling above the microarray surface 2702 and between the second pair of unsealed wells, one of which contains a single inactive microparticle 2704. Pooling of the substrate between the second pair of unsealed wells can further cause noise or interference to be introduced by blocking other fluorescent signals from adjacent wells. For example, and not limitation, pooling of the substrate can cause the resulting image of the array to be hazy or cloudy, which can be due at least in part to fluorescent interference from adjacent wells and/or from unbound material in the substrate, which can prevent or inhibit resolution of whether microparticle 2704 is inactive. For purpose of illustration and not limitation, as embodied herein, the microarray 2702 having sealed wells can decrease noise in the detected image of the active and inactive microparticles 2704 in the array, thereby improving the signal-to-noise ratio of the detection system.

While the disclosed subject matter is described herein in terms of certain preferred embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. An assay surface (AS) for analysis of an analyte of interest in a sample, comprising:
a sample processing component configured to process the sample for detection, wherein the sample processing component comprises a plurality of sample preparation regions, including at least one wash region configured to hold a volume of liquid and at least one storage region configured to hold a plurality of solid supports, wherein the plurality of solid supports is moveable through the plurality of sample preparation regions under a magnetic force; and
a detection component configured to receive the plurality of solid supports by the magnetic force and to detect a presence of the analyte or determine a level or concentration of the analyte.

2. The AS of claim 1, wherein the plurality of solid supports comprises magnetic or paramagnetic microparticles or beads.

3. The AS of claim 1, wherein at least one of the plurality of solid supports specifically binds to the analyte of interest or at least one reagent or conjugate.

4. The AS of claim 1, wherein the sample processing component further comprises the plurality of solid supports in the at least one storage region.

5. The AS of claim 1, wherein the sample processing component further comprises at least one mixing region configured to mix the plurality of solid supports, the analyte of interest, and at least one reagent or conjugate.

6. The AS of claim 5, wherein the sample processing component further comprises the at least one reagent or conjugate in the at least one mixing region.

7. The AS of claim 5, wherein the at least one mixing region has a volume capacity of about 25 μL or less.

8. The AS of claim 5, wherein at least one reagent is selected from a group consisting of a detectable label, a binding member, a dye, a surfactant, a diluent, and a combination thereof.

9. The AS of claim 8, wherein the binding member comprises a receptor or an antibody.

10. The AS of claim 5, wherein the at least one wash region is configured to wash off any molecules not bound to any solid supports.

11. The AS of claim 10, wherein the at least one wash region has a volume capacity of about 10 μL or less.

12. The AS of claim 1, wherein the assay surface comprises a plurality of channels, wherein each of the plurality of channels is in between a first and second sample preparation regions.

13. The AS of claim 12, wherein the assay surface comprises a plurality of stopping elements, wherein at least one of the plurality of stopping elements is between the first and second sample preparation regions.

14. The AS of claim 13, wherein when the at least one stopping element is removed, a volume of liquid in the first region is fluidically connected to a volume of liquid in the second region.

15. The AS of claim 10, wherein after passing the at least one wash region, the plurality of solid supports is moved into the detection component under the magnetic force.

16. The AS of claim 1, wherein the detection component is configured for optical detection, analog detection, or digital detection.

17. The AS of claim 1, wherein the detection component comprises an array of elements, wherein each element of the array of elements is dimensioned to hold a single one of the plurality of solid supports.

18. The AS of claim 17, wherein the array of elements comprises an array of nanowells.

19. The AS of claim 1, wherein the detection component is further configured to receive a substrate fluid, the substrate fluid configured to provide a detectable signal from the plurality of solid supports, and wherein the detection component is further configured to receive an inert liquid, wherein the inert liquid is configured to seal each element of the array of elements containing a portion of the substrate fluid and a single one of the plurality of solid supports.

20. The AS of claim 19, wherein the inert liquid comprises an oil.

21. The AS of claim 1, wherein after the plurality of solid supports is moved into the detection component, the detection component is configured to obtain images of the array of elements.

22. The AS of claim 1, wherein the detection component is configured for single-molecule counting.

23. The AS of claim 1, wherein the assay surface comprises a hydrophobic material.

24. The AS of claim 1, wherein the assay surface further comprises a plurality of volumes of liquids, a plurality of solid supports, and at least one reagent or conjugate in the plurality of sample preparation regions.

25. An assay processing unit (APU) for performing sample processing and analyte detection on an assay surface comprising a sample processing component and a detection component, comprising:
an assay surface receiving component configured to receive and hold an assay surface;
a magnetic element configured to generate a magnetic field, wherein the magnetic field is movable along the assay surface when received by the receiving component; and
one or more processors configured to move the magnetic field to urge at least one solid support disposed on the assay surface through at least one volume of liquid in at least one region of the sample processing component and to the detection component of the assay surface using the magnetic field.

26. The APU of claim 25, wherein the magnetic element comprises a magnet.

27. The APU of claim 26, further comprises a sliding element configured to move the magnetic element under the control of the one or more processors along a horizontal direction of a plane defined by a top surface of the assay surface when received by the receiving component.

28. The APU of claim 27, wherein the sliding element comprises a motor.

29. The APU of claim 26, further comprises a drive element configured to move the magnetic element under the control of the one or more processors in a perpendicular direction to a plane defined by a top surface of the assay surface when received by the receiving component.

30. The APU of claim 29, wherein the drive element comprises a motor or a string.

31. The APU of claim 25, wherein the magnetic element comprises an electromagnet configured to generate a movable magnetic field.

32. The APU of claim 25, further comprises a mixing dynamics element controlled by the one or more processors configured to cause at least one volume of liquid in at least one region of the assay surface when received by the receiving component to mix under a predetermined frequency.

33. The APU of claim 32, wherein the mixing dynamics element comprises a vibration motor or an electromagnet.

34. The APU of claim 25, wherein the one or more processors controls the detection component of the assay surface when received by the receiving component to obtain images of the detection component.

35. An assay processing system (APS) for analysis of an analyte of interest in a sample, comprising:
one or more assay surfaces, wherein at least one assay surface comprises:
a sample processing component configured to process the sample for detection, wherein the sample processing component comprises a plurality of sample preparation regions, including at least one wash region configured to hold a volume of liquid and at least one storage region configured to hold a plurality of solid supports, wherein the plurality of solid supports is moveable through the plurality of sample preparation regions under a magnetic force; and
a detection component configured to receive the plurality of solid supports by the magnetic force and to detect a presence of the analyte or determine a level or concentration of the analyte; and
an assay processing unit (APU) comprising:
an assay surface receiving component configured to receive and hold the one or more assay surfaces;
a magnetic element configured to generate a magnetic field, wherein the magnetic field is movable along at least one assay surface when received by the receiving component;
one or more processors configured to move the magnetic field to urge at least one solid support disposed on the at least one assay surface through at least one volume of liquid in at least one region of the sample processing component and to the detection component of the assay surface using the magnetic field.

36. The APS of claim 35, comprises at least one assay surface according to any of claims 1-24.

37. The APS of claim 35, comprises at least one APU according to any of claims 25-34.

38. A laboratory system for analysis of one or more analytes of interest in a plurality of samples, comprising:
one or more assay processing systems (APSs), wherein at least one APS comprises:
one or more assay surfaces, wherein at least one assay surface comprises:
a sample processing component configured to process the sample for detection, wherein the sample processing component comprises a plurality of sample preparation regions, including at least one wash region configured to hold a volume of liquid and at least one storage region configured to hold a plurality of solid supports, wherein the plurality of solid supports is moveable through the plurality of sample preparation regions under a magnetic force; and
a detection component configured to receive the plurality of solid supports by the magnetic force and to detect a presence of the analyte or determine a level or concentration of the analyte; and
an assay processing unit (APU) comprising:
an assay surface receiving component configured to receive and hold the one or more assay surfaces;
a magnetic element configured to generate a magnetic field, wherein the magnetic field is movable along at least one assay surface when received by the receiving component;
one or more processors configured to move the magnetic field to urge at least one solid support disposed on the at least one assay surface through at least one volume of liquid in at least one region of the sample processing component and to the detection component of the assay surface using the magnetic field; and
a controller configured to control a plurality of the one or more APSs to process a corresponding sample and to detect a presence of at least one corresponding analyte or determine a level or concentration of the at least one corresponding analyte substantially in parallel.

39. The laboratory system of claim 33, comprises at least one assay surface comprising an assay surface according to any of claims 1-24.

40. The laboratory system of claim 33, comprises at least one APU comprising an APU according to any of claims 25-34.

41. The laboratory system of claim 38, wherein the laboratory system is configured to perform one or more of an HIV p24 assay, an HBsAg assay, a Troponin I assay, a TSH assay, a Myoglobobin assay, a PSA assay, a BNP assay, a PIVKA-II assay, an HIV Ab assay, an estradiol assay, and a COVID-Ag assay.

42. The laboratory system of claim 38, wherein the laboratory system has a throughput of at least 360 samples per hour.

43. The laboratory system of any of claim 38, wherein the laboratory system has a throughput of at least 375 of the samples per hour per square meter footprint of the laboratory system.

44. A method for analysis of an analyte of interest in a sample, comprising:
loading at least one volume of liquid into at least one wash region of an assay surface, wherein the assay surface comprising:
a sample processing component configured to process the sample for detection, wherein the sample processing component comprises a plurality of sample preparation regions, including the at least one wash region configured to hold a volume of liquid and at least one storage region configured to hold a plurality of solid supports, wherein the plurality of solid supports is moveable through the plurality of sample preparation regions under a magnetic force; and
a detection component configured to receive the plurality of solid supports by the magnetic force and to detect a presence of the analyte or determine a level or concentration of the analyte;
loading at least one volume of liquid into the detection component;
loading a volume of liquid comprising the analyte into the sample processing component; and
detecting the analyte of interest in the detection component.

45. The method of claim 44, wherein the sample processing component comprises a plurality of solid supports, the method further comprises moving the plurality of solid supports through the plurality sample preparation regions into the detection component under the magnetic force before detecting the analyte of interest in the detection component.

46. The method of claim 44, further comprises:
loading a plurality of solid supports onto the sample processing component,
moving the plurality of solid supports through the plurality sample preparation regions into the detection component under the magnetic force before detecting the analyte of interest in the detection component.

47. The method of claim 44, wherein the assay surface comprises an assay surface according to any of claims 1-24.

48. A method for performing sample processing and analyte detection on an assay surface comprising a sample processing component and a detection component using an assay processing unit (APU), comprising:
receiving an assay surface into an assay surface receiving component of the APU;
generating a magnetic field by a magnetic element of the APU, wherein the magnetic field is movable along the assay surface; and
detecting the analyte of interest in the detection component controlled by the one or more processors of the APU.

49. The method of claim 48, wherein the assay surface comprises a plurality of solid supports, the method further comprises moving the magnetic field controlled by one or more processors of the APU to urge at least one solid support disposed on the assay surface through at least one volume of liquid in at least one region of the sample processing component and to the detection component of the assay surface using the magnetic field before detecting the analyte of interest in the detection component.

50. The method of claim 48, further comprises:
loading a plurality of solid supports onto the sample processing component,
moving the magnetic field controlled by one or more processors of the APU to urge at least one solid support disposed on the assay surface through at least one volume of liquid in at least one region of the sample processing component and to the detection component of the assay surface using the magnetic field before detecting the analyte of interest in the detection component.

51. The method of claim 48, wherein the assay surface comprises an assay surface according to any of claims 1-24.

52. The method of claim 48, wherein the APU comprises an APU according to any of claims 25-34.

53. A method for analysis of an analyte of interest in a sample using an assay processing system (APS) comprising one or more assay surfaces and an assay processing unit (APU), comprising:
loading at least one volume of liquid into at least one wash region of at least one assay surface, wherein the at least one assay surface comprising:
a sample processing component configured to process the sample for detection, wherein the sample processing component comprises a plurality of sample preparation regions, including the at least one wash region configured to hold a volume of liquid and at least one storage region configured to hold a plurality of solid supports, wherein the plurality of solid supports is moveable through the plurality of sample preparation regions under a magnetic force; and
a detection component configured to receive the plurality of solid supports by the magnetic force and to detect a presence of the analyte or determine a level or concentration of the analyte;
loading at least one volume of liquid into the detection component;
loading a volume of liquid comprising the analyte into the sample processing component;
receiving the at least one assay surface into an assay surface receiving component of the APU;
generating a magnetic field by a magnetic element of the APU, wherein the magnetic field is movable along the assay surface; and
detecting the analyte of interest in the detection component controlled by one or more processors of the APU.

54. The method of claim 53, wherein the at least one assay surface comprises a plurality of solid supports, the method further comprises moving the magnetic field controlled by the one or more processors of the APU to urge at least one solid support disposed on the assay surface through at least one volume of liquid in at least one region of the sample processing component and to the detection component of the assay surface using the magnetic field before detecting the analyte.

55. The method of claim 53, further comprises:
loading a plurality of solid supports onto the assay surface, and
moving the magnetic field controlled by the one or more processors of the APU to urge at least one solid support disposed on the assay surface through at least one volume of liquid in at least one region of the sample processing component and to the detection component of the assay surface using the magnetic field before detecting the analyte.

56. The method of claim 53, wherein the assay surface comprises an assay surface according to any of claims 1-24.

57. The method of claim 53, wherein the APU comprises an APU according to any of claims 25-34.

58. A method of using a laboratory system for analysis of one or more analytes of interest in a plurality of samples, wherein the laboratory system comprises one or more assay processing systems (APSs) and a controller, wherein at least one APS comprises one or more assay surfaces and an assay processing unit (APU), the method comprising:
loading at least one volume of liquid into at least one wash region of at least one assay surface, wherein the at least one assay surface comprising:
a sample processing component configured to process the sample for detection, wherein the sample processing component comprises a plurality of sample preparation regions, including the at least one wash region configured to hold a volume of liquid and at least one storage region configured to hold a plurality of solid supports, wherein the plurality of solid supports is moveable through the plurality of sample preparation regions under a magnetic force; and
a detection component configured to receive the plurality of solid supports by the magnetic force and to detect a presence of the analyte or determine a level or concentration of the analyte;
loading at least one volume of liquid into the detection component;
loading a volume of liquid comprising the analyte into the sample processing component;
receiving the at least one assay surface into an assay surface receiving component of a corresponding APU;
generating a magnetic field by a magnetic element of the APU, wherein the magnetic field is movable along the at least one assay surface; and
detecting the analyte of interest in the detection component controlled by one or more processors of the corresponding APU,
wherein the controller is configured to control a plurality of the one or more APSs to perform corresponding steps for a corresponding sample and to detect a presence of at least one corresponding analyte or determine a level or concentration of the at least one corresponding analyte substantially in parallel.

59. The method of claim 58, wherein the at least one assay surface comprises a plurality of solid supports, the method further comprises moving the magnetic field controlled by the one or more processors of the APU to urge at least one solid support disposed on the assay surface through at least one volume of liquid in at least one region of the sample processing component and to the detection component of the assay surface using the magnetic field before detecting the analyte.

60. The method of claim 58, further comprises:
loading a plurality of solid supports onto the at least one assay surface, and
moving the magnetic field controlled by the one or more processors of the APU to urge at least one solid support disposed on the assay surface through at least one volume of liquid in at least one region of the sample processing component and to the detection component of the assay surface using the magnetic field before detecting the analyte.

61. The method of claim 58, wherein at least one assay surface comprises an assay surface according to any of claims 1-24.

62. The method of claim 58, wherein at least one APU comprises an APU according to any of claims 25-34.

63. The method of claim 58, wherein the laboratory system is configured to perform one or more of an HIV p24 assay, an HBsAg assay, a Troponin I assay, a TSH assay, a Myoglobobin assay, a PSA assay, a BNP assay, a PIVKA-II assay, an HIV Ab assay, an estradiol assay, and a COVID-Ag assay.

64. The method of claim 58, wherein the laboratory system has a throughput of at least 360 samples per hour.

65. The method of claim 58, wherein the laboratory system has a throughput of at least 375 of the samples per hour per square meter footprint of the laboratory system.

66. A laboratory system for high-throughput analysis of an analyte of interest in a sample, comprising:
a sample processing component configured to process a sample for detection, wherein the sample processing component is configured to obtain a level or a concentration of an analyte in the sample, or a level or a concentration of a conjugate indicative of the analyte in the sample, suitable for detection; and
a detection component configured to detect a presence of the analyte in the sample;
wherein the laboratory system has a time-to-result of less than 6 minutes, or a time-to-result within a range of 3 to 5 minutes or a time-to-result within a range of 3 to 7 minutes.

67. A laboratory system for high-throughput analysis of an analyte of interest in a sample, comprising:
a sample processing component configured to process a sample for detection, wherein the sample processing component is configured to obtain a level or a concentration of an analyte in the sample, or a level or a concentration of a conjugate indicative of the analyte in the sample, suitable for detection; and
a detection component configured to detect a presence of the analyte in the sample;
wherein the laboratory system has a throughput of at least 360 samples per hour.

68. A laboratory system for high-throughput analysis of an analyte of interest in a sample, comprising:
a sample processing component configured to process a sample for detection, wherein the sample processing component is configured to obtain a level or a concentration of an analyte in the sample, or a level or a concentration of a conjugate indicative of the analyte in the sample, suitable for detection; and
a detection component configured to detect a presence of the analyte in the sample;
wherein the laboratory system has a throughput of at least 375 of the samples per hour per square meter footprint of the laboratory system, or within a range of 375 to 600 samples per hour per square meter footprint of the laboratory system.

69. A method for high-throughput analysis of an analyte of interest in a sample, comprising:
processing a sample for detection, including obtaining a level or a concentration of an analyte in the sample, or a level or a concentration of a conjugate indicative of the analyte in the sample, suitable for detection; and
detecting a presence of the analyte in the sample;
wherein processing the sample and detecting the presence of the analyte in the sample are completed for the sample in less than 6 minutes, or within a range of 3 to 5 minutes, or within a range of 3 to 7 minutes.

70. A method for high-throughput analysis of an analyte of interest in a sample, comprising:
processing a sample for detection, including obtaining a level or a concentration of an analyte in the sample, or a level or a concentration of a conjugate indicative of the analyte in the sample, suitable for detection; and
detecting a presence of the analyte in the sample;
wherein processing the sample and detecting the presence of the analyte in the sample are completed for at least 360 samples per hour.

71. A method for high-throughput analysis of an analyte of interest in a sample, comprising:
processing a sample for detection, including obtaining a level or a concentration of an analyte in the sample, or a level or a concentration of a conjugate indicative of the analyte in the sample, suitable for detection; and
detecting a presence of the analyte in the sample;
wherein processing the sample and detecting the presence of the analyte in the sample are completed for at least 375 of the samples per hour per square meter of the laboratory system, or within a range of 375 to 600 samples per hour per square meter footprint of the laboratory system.
